(19) United States
(12) Patent Application Publication
(10) Pub. No.: US 2010/0331368 A1
(43) Pub. Date: Dec. 30, 2010

(54) 2,5-DIARYL SELENOPHENE COMPOUNDS, AZA 2,5-DIARYL THIOPHENE COMPOUNDS, AND THEIR PRODRUGS AS ANTIPROTOZOAL AGENTS

(76) Inventors: Richard R. Tidwell, Pittsboro, NC (US); David W. Boykin, Atlanta, GA (US); Chad E. Stephens, Augusta, GA (US); Mohamed A. Ismail, Mansoura (EG); Arvind Kumar, Lilburn, GA (US); W. David Wilson, Atlanta, GA (US); Reto Brun, Therwil (CH); Karl Werbovetz, Worthington, OH (US)

Correspondence Address: JENKINS, WILSON, TAYLOR & HUNT, P. A., 3100 Tower Blvd., Suite 1200, DURHAM, NC, 27707, US

(21) Appl. No.: 12/680,844

(22) PCT Filed: Oct. 17, 2008
(86) PCT No.: PCT/US08/11870

§ 371 Date: Sep. 13, 2010

Related U.S. Application Data
(60) Provisional application No. 60/999,262, filed on Oct. 17, 2007.

(51) Int. Cl. A61K 31/444; C07D 345/00; A61K 31/33; A61K 31/4427; C07D 421/04; C07D 421/14; C07D 409/14; A61P 31/00; A61P 33/02

(52) U.S. Cl. 514/333; 540/1; 514/183; 514/336; 546/268.1; 546/256

(57) ABSTRACT

Novel dicationic 2,5-diaryl selenophene compounds are described. Also described are novel aza analogues of dicationic 2,5-diaryl thiophenes. The presently disclosed dicationic compounds exhibit in vitro activity versus Trypanosoma brucei rhodesiense, Plasmodium falciparum, and/or Leishmania donovani comparable to that of pentamidine and furamidine. Some of the novel dicationic compounds display good activity in vivo in a murine model of a Trypanosoma brucei rhodesiense infection.

### RELATED APPLICATIONS

This application claims the benefit of U.S. Patent Application Ser. No. 60/999,262, filed Oct. 17, 2007, the disclosure of which is incorporated herein by reference in its entirety.

### TECHNICAL FIELD

The present invention relates to methods of combating microbial infections with dicationic compounds. More particularly, the present invention relates to methods of combating protozoal infections with dicationic 2,5-diaryl selenophene and aza 2,5-diaryl thiophene compounds, and to the dicationic compounds themselves.

### ABBREVIATIONS

Ac=acetyl
AcOH=acetic acid
Bu=butyl
° C.=degrees Celsius
Calcd.=calculated
CDI=N,N-carbonyldiimidazole
CHCl₃=chloroform
cm=centimeters
dec.=decomposition point
DME=1,2-dimethoxyethane
DMF=dimethylformamide
DMSO=dimethylsulfoxide
D₂O=deuterium oxide
eq.=equivalents
ESI=electrospray ionization
EtOAc=ethyl acetate
EtOH=ethanol
g=grams
h=hours
HAT=human African trypanosomiasis
HCl=hydrogen chloride
HPLC=high-pressure liquid chromatography
Hz=hertz
ip=intraperitoneal
IR=infrared
K₂CO₃=potassium carbonate
kg=kilograms
KO-t-Bu=potassium tert-butoxide
L. d.=Leishmania donovani
LiHMDS=lithium bis(trimethylsilyl)amide
LiOH=lithium hydroxide
M=molar
Me=methyl
MeO=methoxyl
mg=milligrams
MHz=megahertz
min=minutes
mL=milliliters
mm=millimeters
mM=millimolar
mmol=mmoles
mol=moles
mp=melting point
MS=mass spectroscopy
Na₂CO₃=sodium carbonate
NaOH=sodium hydroxide
Na₂SO₄=sodium sulfate
NBS=N-bromosuccinimide
n-BuOH=1-butanol
NH₂OH.HCl=hydroxylamine hydrochloride
NMR=nuclear magnetic resonance
OMe=methoxy
Pd=palladium
Pd(PPh₃)₄=tetrakis(triphenylphosphine)palladium
P. f=Plasmodium falciparum
po=oral
ppm=parts per million
spp.=species
T. br.=Trypanosoma brucei rhodesiense
T. cruzi=Trypanosoma cruzi
THF=tetrahydrofuran
TLC=thin-layer chromatography
TMS=tetramethylsilane
UV=ultraviolet

### BACKGROUND

The incidence of microbial infections (e.g., mycobacterial, fungal, and protozoal infections) in the immunocompromised population has significantly increased over the past several years. In particular, Candida species, especially Candida albicans, are often significant pathogens in patients infected with human immunodeficiency virus (HIV). Another pathogen, Pneumocystis carinii, causes a form of pneumonia (PCP) that is believed to be one of the leading causes of death in patients suffering from AIDS.

In addition, human African trypanosomiasis (HAT) has reemerged as a threat to over 60 million people. Current estimates are that between 350,000 and 450,000 people are infected with HAT. Other severe and life-threatening microbial infections are caused by Plasmodium spp., Mycobacterium tuberculosis, Aspergillus spp., Cryptosporidium parvum, Giardia lamblia, Toxoplasma gondii, Fusarium solani, and Cryptococcus neoformans.

The antimicrobial properties of dicationic molecules have been studied since the 1930's. Compounds of this type have typically utilized amidine groups as the cationic moieties, and their activities against a number of pathogens including Trypanosoma spp., Plasmodium spp., Cryptosporidium parvum, Giardia lamblia, Leishmania spp., Pneumocystis carinii, Toxoplasma gondii, Candida albicans, Aspergillus spp. and Cryptococcus neoformans have been reported. See, e.g., King, H., et al., Ann. Trop. Med. Parasitol., 32, 177-192 (1938); Blagburn, B. L., et al., Antimicrob. Agents Chemother., 35, 1520-1523 (1991); Bell, C. A., et al., Antimicrob. Agents Chemother., 35, 1099-1107 (1991); Bell, C. A., et al., Antimicrob. Agents Chemother., 34, 1381-1386 (1990); Kirk, R., et al., Ann. Trop. Med. Parastiol., 34, 181-197 (1940); Fulton, J. D., Ann. Trop. Med. Parasitol., 34, 53-66 (1940); Ivady, V. G., et al., Monatschr. Kinderheilkd., 106, 10-14 (1958); Boykin, D. W., et al., J. Med. Chem., 38, 912-916 (1995); Boykin, D. W., et al., J. Med. Chem., 41, 124-129 (1998); Francesconi, I., et al., J. Med. Chem., 42, 2260-2265 (1999); Lindsay, D. S., et al., Antimicrob. Agents Chemother., 35, 1914-1916 (1991); Lourie, E. M. et al., Ann. Trop. Med. Parasitol., 33, 289-304 (1939); Lourie, E. M., et al., Ann. Trop. Med. Parasitol., 33, 305-312 (1939); Das, B. P., et al., J. Med. Chem., 20, 531-536 (1976); Del Poeta, M., et al., J. Antimicrob. Chemother., 44, 223-228 (1999); Del Poeta, M., et al., Antimicrob. Agents Chemother., 42, 2495-2502 (1998); Del Poeta, M., et al., Antimicrob. Agents Chemother., 42, 2503-2510 (1998).

Despite the broad-spectrum of antimicrobial activity exhibited by aromatic diamidines, few compounds of this chemical type have seen significant clinical use in humans. Pentamidine has been used clinically against African trypanosomiasis, antimony-resistant leishmaniasis, and P. carinii pneumonia. See, e.g., Apted, F. I. C., Pharmacol. Ther., 11, 391-413 (1980); Bryceson, A. D. M., et al., Trans. Roy. Soc. Trop. Med. Hyg., 79, 705-714 (1985); Hughes, W. T., et al., Antimicrob. Agents Chemother., 5, 289-293 (1974). Pafuramidine maleate (a prodrug of furamidine) is currently in clinical trials against PCP and malaria. See Barrett, M. P., et al., British J. Pharmacology, 1-17 (2007).

Thus, there continues to be a need for additional compounds having desirable anti-microbial activity, whether against the representative pathogens referenced above or against other pathogens. There also exists a need for orally available prodrugs for treating microbial infections.

### SUMMARY

In some embodiments, the presently disclosed subject matter provides a compound of Formula (I):

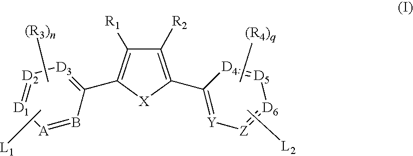

wherein:

X is Se or S;

D₁, D₂, D₃, D₄, D₅, and D₆ are each C or N;

A, B, Y, and Z are each independently selected from the group consisting of CH and N, provided that when X is S, at least two of A, B, Y, and Z are N;

n is an integer from 0 to 2;

q is an integer from 0 to 2;

R₁ and R₂ are independently selected from the group consisting of H, alkyl, substituted alkyl, cycloalkyl, aryl, substituted aryl, aralkyl, halogen, alkoxyl, aryloxyl, aralkoxyl, and hydroxyl;

each R₃ and R₄ is independently selected from the group consisting of H, alkyl, substituted alkyl, cycloalkyl, aryl, substituted aryl, aralkyl, halogen, alkoxyl, aryloxyl, aralkoxyl, and hydroxyl; and

L₁ and L₂ are independently selected from the group consisting of:

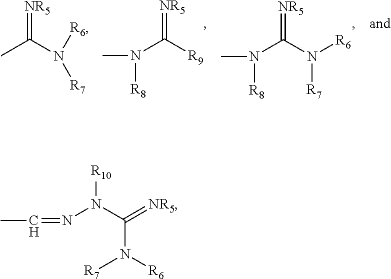

wherein:

each R₅ is selected from the group consisting of H, hydroxyl, alkyl, cycloalkyl, aryl, aralkyl, alkoxyl, aryloxyl, hydroxycycloalkyl, alkoxycycloalkyl, hydroxyalkyl, aminoalkyl, acyloxyl, and alkylaminoalkyl; and

each R₆, R₇, R₈, R₉, and R₁₀ is independently selected from the group consisting of H, alkyl, alkoxyalkyl, cycloalkyl, aryl, aralkyl, hydroxyalkyl, aminoalkyl, and alkylaminoalkyl, or a R₅ and a R₆ or a R₅ and a R₉ together represent a C₂ to C₁₀ alkylene, or a R₅ and a R₆ or a R₅ and a R₉ together are:

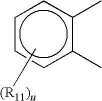

wherein u is an integer from 1 to 4, and R₁₁ is H or —CONHR₁₂NR₁₃R₁₄, wherein R₁₂ is alkylene and R₁₃ and R₁₄ are each independently selected from the group consisting of H, alkyl, aryl and aralkyl; or

a pharmaceutically acceptable salt thereof.

In some embodiments, D₁, D₂, D₃, D₄, D₅, and D₆ are each C and the compound of Formula (I) has a structure of Formula (Ia):

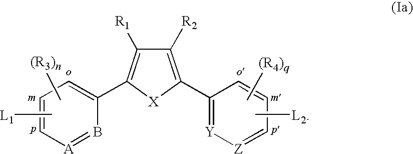

In some embodiments, L₁ is attached at the p carbon and L₂ is attached at the p′ carbon.

In some embodiments, L₁ and L₂ are each:

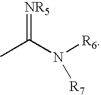

In some embodiments, R₅ is selected from the group consisting of H, hydroxyl, alkoxyl, and acyloxyl. In some embodiments, each R₅ is selected from H, hydroxyl, methoxyl, and N,N-dimethylaminoacetoxyl. In some embodiments R₆ and R₇ are each H.

In some embodiments, X is Se and the compound of Formula (I) has a structure of Formula (II):

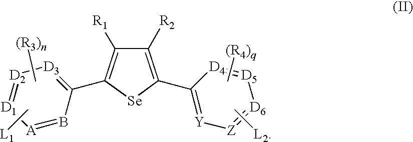

In some embodiments, A, B, Y, and Z are each CH. In some embodiments, at least one of A, B, Y, and Z are N. In some embodiments, A and Z are N. In some embodiments, B and Y are N.

In some embodiments, X is S and the compound of Formula (I) has a structure of Formula (III):

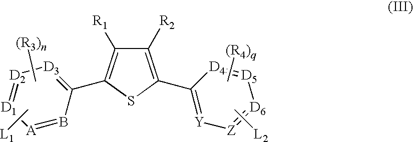

wherein at least two of A, B, Y and Z are N.

In some embodiments, the compound has a structure of one of Formula (IIIb) and Formula (IIIc):

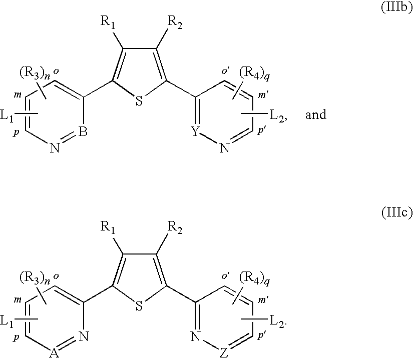

In some embodiments, the compound of Formula (I) is selected from the group consisting of:
2,5-Bis(4-amidinophenyl)selenophene;
2,5-Bis[4-(N-hydroxyamidino)phenyl]selenophene;
2,5-Bis[4-(N-methoxyamidino)phenyl]selenophene;
6-[5-(4-amidinophenyl)-selenophen-2-yl]-nicotinamidine;
6-{5-[4-(N-hydroxyamidino)phenyl]-selenophen-2-yl}-N-hydroxynicotinamidine;
6-{5-[4-(N-methoxyamidino)phenyl]-selenophen-2-yl}-N-methoxynicotinamidine;
5-[5-(4-amidinophenyl)-selenophene-2-yl]-pyridine-2-amidine;
5-{5-[4-(N-hydroxyamidino)-phenyl]-selenophen-2-yl}-pyridine-2-N-hydroxyamidine;
5-{5-[4-(N-methoxyamidino)-phenyl]-selenophen-2-yl}-pyridine-2-N-methoxyamidine;
2,5-Bis-[5-(2-amidinopyridyl)]selenophene;
2,5-Bis-[5-{2-(N-hydroxyamidino)-pyridyl}]selenophene;
2,5-Bis-[5-{2-(N-methoxyamidino)-pyridyl}]selenophene;
2,5-Bis-[5-{2-N—(N,N-dimethylaminoacetoxyamidino)-pyridyl}]selenophene;
2,5-Bis-[2-(5-amidinopyridyl)]selenophene;
2,5-Bis-[2-{5-(N-hydroxyamidino)-pyridyl}]selenophene;
2,5-Bis-[2-{5-(N-methoxyamidino)pyridyl}]selenophene;
2,5-Bis-[2-(5-amidinopyridyl)]thiophene;
2,5-Bis-[2-{5-(N-hydroxyamidino)-pyridyl}]thiophene;
2,5-Bis-[2-{5-(N-methoxyamidino)-pyridyl}]thiophene;
2,5-Bis-[5-(2-amidinopyridyl)]thiophene;
2,5-Bis-[5-{2-(N-hydroxyamidino)-pyridyl}]thiophene; and
2,5-Bis-[5-{2-(N-methoxyamidino)-pyridyl}]thiophene;
or a pharmaceutically acceptable salt thereof.

In some embodiments, the pharmaceutically acceptable salt is a hydrochloride salt.

In some embodiments, the presently disclosed subject matter provides a pharmaceutical formulation comprising a compound of Formula (I) and a pharmaceutically acceptable carrier.

In some embodiments, the presently disclosed subject matter provides a method of treating a microbial infection in a subject in need of treatment thereof, the method comprising administering to the subject an effective amount of a compound of Formula (I). In some embodiments, the microbial infection is selected from the group consisting of a Trypanosoma brucei rhodesiense infection, a Plasmodium falciparum infection, and a Leishmania donovoni infection. In some embodiments, the compound of Formula (I) is administered prophylactically to prevent or reduce the incidence of one of a microbial infection in a subject at risk of infection, a recurrence of a microbial infection, and a combination thereof.

It is accordingly an object of the presently disclosed subject matter to provide methods and compositions for treating microbial infections, such as, but not limited to, those caused by Trypanosoma species (spp.), including, but not limited to, Trypanosoma brucei rhodesiense, Trypanosoma brucei gambiense, Trypanosoma brucei brucei, and Trypanosoma cruzi; Plasmodium spp., including but not limited to Plasmodium falciparum; and Leishmania spp., including but not limited to Leishmania donovani and Leishmania mexicana amazonensis, in a subject in need thereof.

An object of the presently disclosed subject matter having been stated hereinabove, which is addressed in whole or in part by the presently disclosed subject matter, other objects and aspects will become evident as the description proceeds when taken in connection with the accompanying Examples as best described herein below.

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist.

### I. DEFINITIONS

Following long-standing patent law convention, the terms “a” and “an” mean “one or more” when used in this application, including the claims. Thus, for example, “a compound” can refer to two or more different compounds.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl)hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with one or more atoms or functional groups, including for example, alkyl, substituted alkyl, halogen, e.g., —CH₂X, —CHX₂, and —CX₃, wherein X is a halogen selected from the group consisting of Cl, Br, F, and I, aryl, substituted aryl, alkoxyl, hydroxyl, nitro, amino, alkylamino, dialkylamino, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

As used herein, the term “aza” refers to a heterocyclic ring structure containing at least one nitrogen atom. The term “azaaryl” refers to an aromatic group having a heterocyclic ring structure containing at least one nitrogen atom. Specific examples of azaaryl groups include, but are not limited to, pyrrolidine, piperidine, quinuclidine, pyridine, pyrrole, indole, purine, pyridazine, pyrimidine, and pyrazine. In some embodiments, azaaryl refers to a pyridine or pyridazine group.

A structure represented generally by a formula such as:

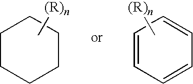

as used herein refers to a ring structure, for example, but not limited to a 3-carbon, a 4-carbon, a 5-carbon, a 6-carbon, and the like, aliphatic and/or aromatic cyclic compound comprising a substituent R group, wherein the R group can be present or absent, and when present, one or more R groups can each be substituted on one or more available carbon atoms of the ring structure, replacing an H atom that would be bonded to that carbon in the absence of the R group. The presence or absence of the R group and the number of R groups is determined by the value of the integer n. Each R group, if more than one, is substituted on an available carbon of the ring structure rather than on another R group. For example, the structure:

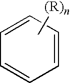

wherein n is an integer from 0 to 2 comprises compound groups including, but not limited to:

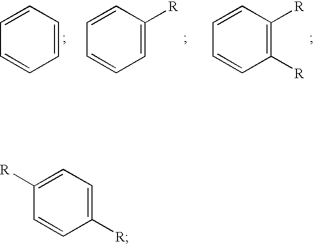

and the like.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

As used herein, the term “acyl” refers to a carboxylic acid group wherein the —OH of the carboxylic acid group has been replaced with another substituent. Thus, an acyl group can be represented by RC(═O)—, wherein R is an alkyl, substituted alkyl, aryl, or substituted aryl group as defined herein. In some embodiments, the term acyl can refer to the group RC(═O)— wherein R is an amino-substituted alkyl group, an alkylamino-substituted alkyl group, a dialkylamino-substituted alkyl group, or a hydroxyl-substituted alkyl group. Thus, the term “acyl” can refer to groups such as H₂NRC(═O)—, R′HNRC(═O)—, R″R′NRC(═O)—, and HORC(═O)—, wherein R is alkylene and R′ and R″ are lower alkyl. These acyl groups can also be referred to as “aminoalkylcarbonyl,” “alkylaminoalkylcarbonyl,” “dialkylaminoalkylcarbonyl,” and “hydroxyalkylcarbonyl” groups. The term “acyl” also specifically includes arylacyl groups, such as an acetylfuran and a phenacyl group. Arylacyl groups, (i.e., RC(═O)— groups wherein R is aryl) can also be referred to as “aroyl” groups. Specific examples of acyl groups include acetyl and benzoyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

“Alkoxyl” and “alkyloxyl” refer to an alkyl-O— group wherein alkyl is as previously described, including a substituted alkyl. The term “alkoxyl” as used herein can refer to C₁₋₂₀ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl- group wherein aryl and alkyl are as previously described, and included substituted aryl and substituted alkyl. Exemplary aralkyl groups include benzyl, phenylethyl, and naphthylmethyl.

“Aralkyloxyl” and “aralkoxyl” refer to an aralkyl-O— group wherein the aralkyl group is as previously described. An exemplary aralkyloxyl group is benzyloxyl.

“Dialkylamino” refers to an —NRR′ group wherein each of R and R′ is independently an alkyl group and/or a substituted alkyl group as previously described. Exemplary dialkylamino groups include ethylmethylamino, dimethylamino, and diethylamino. The term “alkylamino” refers to an —NHR group wherein R is an alkyl or substituted alkyl group.

“Alkoxycarbonyl” refers to an alkyl-O—C(═O)— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an H₂N—C(═O)— group.

“Alkylcarbamoyl” refers to a R′RN—C(═O)— group wherein one of R and R′ is hydrogen and the other of R and R′ is alkyl and/or substituted alkyl as previously described.

“Dialkylcarbamoyl” refers to a R′RN—C(═O)— group wherein each of R and R′ is independently alkyl and/or substituted alkyl as previously described.

“Acyloxyl” refers to an acyl—O— group wherein acyl is as previously described. Acyloxyl groups include, but are not limited to, acetoxy groups (i.e., CH₃C(═O)O—). Acyloxyl groups also include aminoalkylcarbonyloxy, alkylaminoalkylcarbonyloxy, dialkylaminoalkylcarbonyloxy, and hydroxyalkylcarbonyloxy groups. In some embodiments, the acyloxyl group has the formula —O—C(═O)—(CH₂)ₓN(R′)s, wherein x is an integer from 1 to 5 and each R′ is independently H or alkyl.

“Acylamino” refers to an acyl-NH— group wherein acyl is as previously described.

“Aroylamino” refers to an aroyl-NH— group wherein aroyl is as previously described.

The term “amino” refers to the —NH₂ group.

The term “carbonyl” refers to the —(C═O)— group.

The term “carboxylic acid” refers to the —C(═O)OH group. The terms “carboxy” and “carboxylate” refer to the —C(═O)O⁻ anion.

The terms “halo”, “halide”, or “halogen” as used herein refer to fluoro, chloro, bromo, and iodo groups.

The term “hydroxyl” refers to the —OH group.

The term “hydroxyalkyl” refers to an alkyl group substituted with an —OH group.

The term “mercapto” refers to the —SH group.

The term “oxo” refers to a compound described previously herein wherein a carbon atom is replaced by an oxygen atom.

The term “nitro” refers to the —NO₂ group.

The term “thio” refers to a compound described previously herein wherein a carbon or oxygen atom is replaced by a sulfur atom.

The term “sulfate” refers to the —SO₄ group.

When the term “independently selected” is used, the substituents being referred to (e.g., R groups, such as groups R₁ and R₂, or groups X and Y), can be identical or different. For example, both R₁ and R₂ can be substituted alkyls, or R₁ can be hydrogen and R₂ can be a substituted alkyl, and the like. A named “R”, “B,” “X,” “Y,” “A,” “D,” “L,” or “Z” group will generally have the structure that is recognized in the art as corresponding to a group having that name, unless specified otherwise herein. For the purposes of illustration, certain representative “R,” “B,” “X,” “Y”, “A,” “D,” “L,” and “A” groups as set forth above are defined below. These definitions are intended to supplement and illustrate, not preclude, the definitions that would be apparent to one of ordinary skill in the art upon review of the present disclosure.

The term “reflux” and grammatical derivations thereof refer to boiling a liquid, such as a solvent, in a container, such as a reaction flask, with which a condenser is associated, thereby facilitating continuous boiling without loss of liquid, due to the condensation of vapors on the interior walls of the condenser.

### II. NOVEL COMPOUNDS

Disclosed herein are compounds of Formula (I):

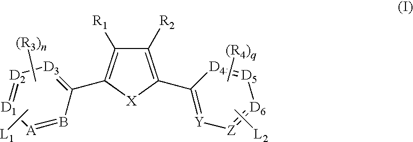

wherein:

X is Se or S;

D₁, D₂, D₃, D₄, D₅, and D₆ are independently C or N;

A, B, Y, and Z are each independently selected from the group consisting of CH and N, provided that when X is S, at least two of A, B, Y, and Z are N;

n is an integer from 0 to 2;

q is an integer from 0 to 2;

R₁ and R₂ are independently selected from the group consisting of H, alkyl, substituted alkyl, cycloalkyl, aryl, substituted aryl, aralkyl, halogen, alkoxyl, aryloxyl, aralkoxyl, and hydroxyl;

each R₃ and R₄ is independently selected from the group consisting of H, alkyl, substituted alkyl, cycloalkyl, aryl, substituted aryl, aralkyl, halogen, alkoxyl, aryloxyl, aralkoxyl, and hydroxyl; and

L₁ and L₂ are independently selected from the group consisting of:

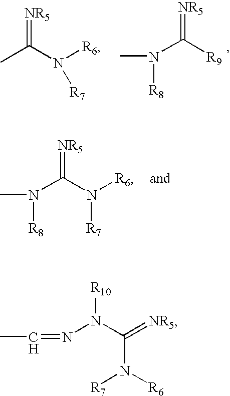

wherein:

each R₅ is selected from the group consisting of H, hydroxyl, alkyl, cycloalkyl, aryl, aralkyl, alkoxyl, aryloxyl, hydroxycycloalkyl, alkoxycycloalkyl, hydroxyalkyl, aminoalkyl, acyloxyl, and alkylaminoalkyl; and

each R₆, R₇, R₈, R₉, and R₁₀ is independently selected from the group consisting of H, alkyl, alkoxyalkyl, cycloalkyl, aryl, aralkyl, hydroxyalkyl, aminoalkyl, and alkylaminoalkyl, or a R₅ and a R₆ or a R₅ and a R₉ together represent a C₂ to C₁₀ alkylene, or a R₅ and a R₆ or a R₅ and a R₉ together are:

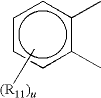

wherein u is an integer from 1 to 4, and R₁₁ is H or —CONHR₁₂NR₁₃R₁₄, wherein R₁₂ is alkylene and R₁₃ and R₁₄ are each independently selected from the group consisting of H, alkyl, aryl and aralkyl; or

a pharmaceutically acceptable salt thereof.

In some embodiments, each of D₁, D₂, D₃, D₄, D₅, and D₆ is carbon (i.e., C) and the compound of Formula (I) is a compound of Formula (Ia):

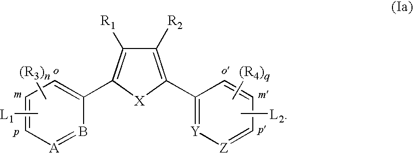

Substituents (e.g., L₁, L₂, R₃, and R₄) attached to the atoms of the two outer aryl groups can be referred to based upon their orientation relative to the central five-membered aryl group. Thus, the R₃, R₄, L₁ and L₂ groups can be referred to as being para, meta, or ortho to the central aryl group. For example, substituents attached to the D₁ or D₆ group of Formula (I) are para to the central aryl group. As indicated in Formula (Ia), the carbon in the D₁ position from the structure of Formula (I) can be referred to as the p carbon, while the carbon in the D₆ position from Formula (I) can be referred to as the p′ carbon. The carbons in the structure of Formula (Ia) that are at the D₂ and D₅ positions from Formula (I) can be referred to as the m carbon and the m′ carbon, respectively. Any substituents attached at the m and m′ carbons of Formula (Ia) can be referred to as being oriented meta to the central five-membered ring. The carbons of Formula (Ia) that are in D₃ and D₄ positions from Formula (I) can be referred to as the o and o′ carbons. Any substituents attached at the o and o′ carbons of Formula (Ia) can be referred to as being oriented ortho to the central five-membered ring.

In some embodiments, L₁ is attached at the p carbon and L₂ is attached at the p′ carbon.

In some embodiments, n and q are each 2. In some embodiments, a R₃ is attached at the m carbon and another R₃ is attached at the o carbon. In some embodiments, a R₄ group is attached at the o′ carbon and another R₄ group is attached at the m′ carbon. In some embodiments, each R₃ and R₄ is H.

In some embodiments, L₁ and L₂ are each:

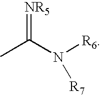

In some embodiments, each R₅ is selected from the group consisting of H, hydroxyl, alkoxyl, and acyloxyl. In some embodiments, the alkoxyl group is selected from methoxyl and ethoxyl. In some embodiments, the acyloxyl group has the formula:

—O—C(═O)—(CH₂)ₓN(R₁₅)₂

wherein x is an integer from 1 to 5; and each R₁₅ is independently H or alkyl. In some embodiments, x is 1 and each R₁₅ is methyl and the acyloxyl group is a N,N-dimethylaminoacetoxyl group (i.e., —O—C(═O)—CH₂—N(CH₃)₂).

In some embodiments, R₆ and R₇ are each H.

In some embodiments, the compound of Formula (I) is selected from the group consisting of: 2,5-Bis(4-amidinophenyl)selenophene; 2,5-Bis[4-(N-hydroxyamidino)phenyl]selenophene; 2,5-Bis[4-(N-methoxyamidino)phenyl]-selenophene; 6-[5-(4-amidinophenyl)selenophen-2-yl]nicotinamidine; 6-{5-[4-(N-hydroxyamidino)phenyl]selenophen-2-yl}-N-hydroxynicotinamidine; 6-{5-[4-(N-methoxyamidino)phenyl]selenophen-2-yl}-N-methoxynicotinamidine; 5-[5-(4-amidinophenyl)selenophene-2-yl]pyridine-2-amidine; 5-{5-[4-(N-hydroxy-amidino)phenyl]selenophen-2-yl}pyridine-2-N-hydroxamidine; 5-{5-[4-(N-methoxyamidino)phenyl]selenophen-2-yl}pyridine-2-N-methoxamidine; 2,5-Bis-[5-(2-amidinopyridyl)]selenophene; 2,5-Bis-[5-{2-(N-hydroxyamidino)pyridyl}]-selenophene; 2,5-Bis-[5-{2-(N-methoxyamidino)pyridyl}]selenophene; 2,5-Bis-[5-{2-N—(N,N-dimethylaminoacetoxyamidino)pyridyl}]selenophene; 2,5-Bis-[2-(5-amidinopyridyl)]selenophene; 2,5-Bis-[2-{5-(N-hydroxyamidino)pyridyl}]-selenophene; 2,5-Bis-[2-{5-(N-methoxyamidino)pyridyl}]selenophene; 2,5-Bis-[2-(5-amidinopyridyl)]thiophene; 2,5-Bis-[2-{5-(N-hydroxyamidino)pyridyl}]-thiophene; 2,5-Bis-[2-{5-(N-methoxyamidino)-pyridyl}]thiophene; 2,5-Bis-[5-(2-amidinopyridyl)]thiophene; 2,5-Bis-[5-{2-(N-hydroxyamidino)pyridyl}]thiophene; and 2,5-Bis-[5-{2-(N-methoxy-amidino)pyridyl}]thiophene.

II.A. 2,5-Diaryl Selenophene Compounds

In some embodiments, X is Se and the compound of Formula (I) is a 2,5-diaryl selenophene compound having a structure of Formula (II):

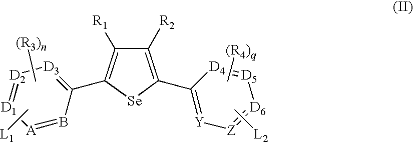

wherein:

D₁, D₂, D₃, D₄, D₅, and D₆ are each C or N;

A, B, Y, and Z are each independently selected from the group consisting of CH and N;

n is an integer from 0 to 2;

q is an integer from 0 to 2;

R₁ and R₂ are independently selected from the group consisting of H, alkyl, substituted alkyl, cycloalkyl, aryl, substituted aryl, aralkyl, halogen, alkoxyl, aryloxyl, aralkoxyl, and hydroxyl;

each R₃ and R₄ is independently selected from the group consisting of H, alkyl, substituted alkyl, cycloalkyl, aryl, substituted aryl, aralkyl, halogen, alkoxyl, aryloxyl, aralkoxyl, and hydroxyl; and

L₁ and L₂ are independently selected from the group consisting of:

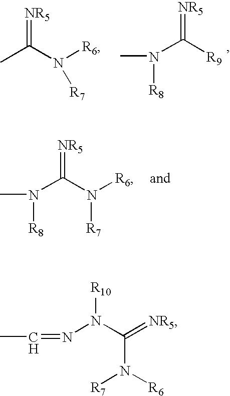

wherein:

each R₅ is selected from the group consisting of H, hydroxyl, alkyl, cycloalkyl, aryl, aralkyl, alkoxyl, aryloxyl, hydroxycycloalkyl, alkoxycycloalkyl, hydroxyalkyl, aminoalkyl, acyloxyl, and alkylaminoalkyl; and

each R₆, R₇, R₈, R₉, and R₁₀ is independently selected from the group consisting of H, alkyl, alkoxyalkyl, cycloalkyl, aryl, aralkyl, hydroxyalkyl, aminoalkyl, and alkylaminoalkyl, or a R₅ and a R₆ or a R₅ and a R₉ together represent a C₂ to C₁₀ alkylene, or a R₅ and a R₆ or a R₅ and a R₉ together are:

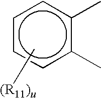

wherein u is an integer from 1 to 4, and R₁₁ is H or —CONHR₁₂NR₁₃R₁₄, wherein R₁₂ is alkylene and R₁₃ and R₁₄ are each independently selected from the group consisting of H, alkyl, aryl and aralkyl; or

a pharmaceutically acceptable salt thereof.

In some embodiments, each of D₁, D₂, D₃, D₄, D₅, and D₆ is C and the compound of Formula (II) has a structure of Formula (IIa):

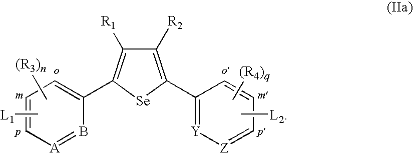

Each R₃, R₄, L₁ and L₂ can be independently attached to their respective six-membered aryl groups in a para, meta, or ortho orientation relative to the selenophene group. For example, in Formula (IIa), L₁ can be attached at one of the p carbon, the m carbon or the o carbon. L₂ can be attached at one of the p′ carbon, the m′ carbon, or the o′ carbon. In some embodiments, L₁ is attached at the p carbon and L₂ is attached at the p′ carbon and the compound of Formula (IIa) has a structure of Formula (IIb):

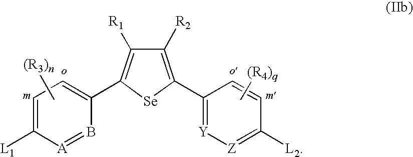

In some embodiments, n and q are each 2, and a R₃ is attached at the m carbon, another R₃ is attached at the o carbon, a R₄ group is attached at the o′ carbon, and another R₄ group is attached at the m′ carbon. In some embodiments, each R₃ and R₄ is H.

In some embodiments, each of A, B, Y and Z is CH and the compound of Formula (II) comprises a dicationic 2,5-diphenyl selenophene compound.

In some embodiments, the compound of Formula (II), (IIa) or (IIb) comprises an aza analogue of a 2,5-diphenyl selenophene compound wherein at least one of A, B, Y, and Z is N. Thus, in some embodiments, the compound of Formula (II), (IIa) or (IIb) comprises one or more pyridine or pyridazine ring attached to a selenophene group.

In some embodiments, the compound of Formula (II) is a 2,5-diazaaryl selenophene. For example, in some embodiments, B and Y are each N. In some embodiments, the compound of Formula (II) has a structure of Formula (IIc):

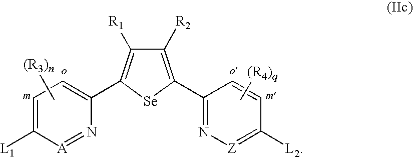

In some embodiments, A and Z are each N. In some embodiments, the compound of Formula (II) has a structure of Formula (IId):

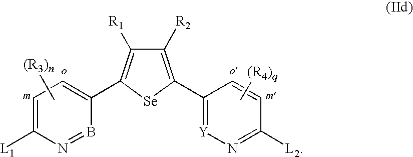

In some embodiments, L₁ and L₂ of the compound of Formula (II), (IIa), (IIb), (IIc) or (IId) are each:

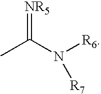

In some embodiments, each R₅ is selected from the group consisting of H, hydroxyl, alkoxyl, and acyloxyl. In some embodiments, the alkoxyl group is selected from methoxyl and ethoxyl. In some embodiments, the acyloxyl group has the formula:

—O—C(═O)—(CH₂)ₓN(R₁₅)₂

wherein x is an integer from 1 to 5; and each R₁₅ is independently H or alkyl. In some embodiments, x is 1 and each R₁₅ is methyl and the acyloxyl group is a N,N-dimethylaminoacetoxyl group.

In some embodiments, R₆ and R₇ are each H.

In some embodiments, the compound of Formula (II) is selected from the group consisting of: 2,5-Bis(4-amidinophenyl)selenophene; 2,5-Bis[4-(N-hydroxyamidino)phenyl]selenophene; 2,5-Bis[4-(N-methoxyamidino)phenyl]-selenophene; 6-[5-(4-amidinophenyl)selenophen-2-yl]nicotinamidine; 6-{5-[4-(N-hydroxyamidino)phenyl]selenophen-2-yl}-N-hydroxynicotinamidine; 6-{5-[4-(N-methoxy-amidino)phenyl]selenophen-2-yl}-N-methoxynicotinamidine; 5-[5-(4-amidino-phenyl)selenophene-2-yl]pyridine-2-amidine; 5-{5-[4-(N-hydroxy-amidino)phenyl]selenophen-2-yl}pyridine-2-N-hydroxamidine; 5-{5-[4-(N-methoxyamidino)phenyl]selenophen-2-yl}-pyridine-2-N-methoxamidine; 2,5-Bis-[5-(2-amidinopyridyl)]selenophene; 2,5-Bis-[5-{2-(N-hydroxyamidino)pyridyl}]-selenophene; 2,5-Bis-[5-{2-(N-methoxyamidino)pyridyl}]selenophene; 2,5-Bis-[5-{2-N—(N,N-dimethylaminoacetoxyamidino)pyridyl}]selenophene; 2,5-Bis-[2-(5-amidinopyridyl)]selenophene; 2,5-Bis-[2-{5-(N-hydroxyamidino)pyridyl}]-selenophene; and 2,5-Bis-[2-{5-(N-methoxyamidino)pyridyl}]selenophene.

II.B. Aza Derivatives of 2,5-Diaryl Thiophene Compounds

In some embodiments, X is S and the compound of Formula (I) has a structure of Formula (III):

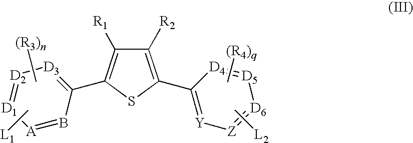

wherein:

D₁, D₂, D₃, D₄, D₅, and D₆ are each C or N;

A, B, Y, and Z are each independently selected from the group consisting of CH and N, provided at least two of A, B, Y, and Z are N;

n is an integer from 0 to 2;

q is an integer from 0 to 2;

R₁ and R₂ are independently selected from the group consisting of H, alkyl, substituted alkyl, cycloalkyl, aryl, substituted aryl, aralkyl, halogen, alkoxyl, aryloxyl, aralkoxyl, and hydroxyl;

each R₃ and R₄ is independently selected from the group consisting of H, alkyl, substituted alkyl, cycloalkyl, aryl, substituted aryl, aralkyl, halogen, alkoxyl, aryloxyl, aralkoxyl, and hydroxyl; and

L₁ and L₂ are independently selected from the group consisting of:

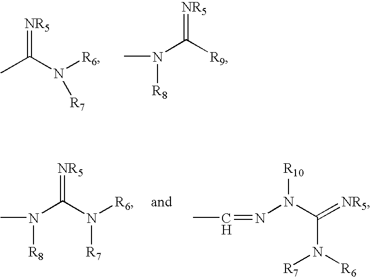

wherein:

each R₅ is selected from the group consisting of H, hydroxyl, alkyl, cycloalkyl, aryl, aralkyl, alkoxyl, aryloxyl, hydroxycycloalkyl, alkoxycycloalkyl, hydroxyalkyl, aminoalkyl, acyloxyl, and alkylaminoalkyl; and

each R₆, R₇, R₈, R₉, and R₁₀ is independently selected from the group consisting of H, alkyl, alkoxyalkyl, cycloalkyl, aryl, aralkyl, hydroxyalkyl, aminoalkyl, and alkylaminoalkyl, or a R₅ and a R₆ or a R₅ and a R₉ together represent a C₂ to C₁₀ alkylene, or a R₅ and a R₆ or a R₅ and a R₉ together are:

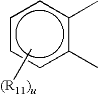

wherein u is an integer from 1 to 4, and R₁₁ is H or —CONHR₁₂NR₁₃R₁₄, wherein R₁₂ is alkylene and R₁₃ and R₁₄ are each independently selected from the group consisting of H, alkyl, aryl and aralkyl; or

a pharmaceutically acceptable salt thereof.

In some embodiments, each of D₁, D₂, D₃, D₄, D₅, and D₆ is C and the compound of Formula (III) has a structure of Formula (IIIa):

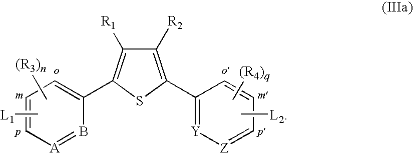

Each R₃, R₄, L₁ and L₂ can be independently attached to their respective six-membered aryl groups in a para, meta, or ortho orientation relative to the thiophene group. For example, L₁ can be attached at one of the p carbon, the m carbon or the o carbon. L₂ can be attached at one of the p′ carbon, the m′ carbon, or the o′ carbon. In some embodiments, L₁ is attached at the p carbon and L₂ is attached at the p′ carbon.

In some embodiments, each of the two six-membered rings of the compound of Formula (III) or (IIIa) comprises at least one ring nitrogen atom and the compound of Formula (III) or Formula (IIIa) is a 2,5-diazaaryl thiophene. In some embodiments, A and Z are both N or B and Y are both N, and the compound of Formula (III) has a structure of one of Formulas (IIIb) and (IIIc):

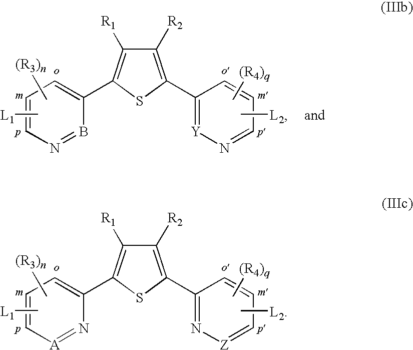

In some embodiments, n and q are each 2, and a R₃ is attached at the m carbon, another R₃ is attached at the o carbon, a R₄ group is attached at the o′ carbon, and another R₄ group is attached at the m′ carbon. In some embodiments, each R₃ and R₄ is H.

In some embodiments, L₁ and L₂ of the compound of Formula (III), (IIIa), (IIIb), or (IIIc) are each:

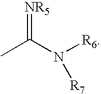

In some embodiments, each R₅ is selected from the group consisting of H, hydroxyl, alkoxyl, and acyloxyl. In some embodiments, the alkoxyl group is selected from methoxyl and ethoxyl. In some embodiments, the acyloxyl group has the formula:

—O—C(═O)—(CH₂)ₓN(R₁₅)₂

wherein x is an integer from 1 to 5; and each R₁₅ is independently H or alkyl. In some embodiments, x is 1 and each R₁₅ is methyl and the acyloxyl group is a N,N-dimethylaminoacetoxyl group.

In some embodiments, R₆ and R₇ are each H.

In some embodiments, the compound of Formula (III) is selected from the group consisting of: 2,5-Bis-[2-(5-amidinopyridyl)]thiophene; 2,5-Bis-[2-{5-(N-hydroxyamidino)-pyridyl}]thiophene; 2,5-Bis-[2-{5-(N-methoxyamidino)-pyridyl}]thiophene; 2,5-Bis-[5-(2-amidinopyridyl)]thiophene; 2,5-Bis-[5-{2-(N-hydroxyamidino)pyridyl}]thiophene); and 2,5-Bis-[5-{2-(N-methoxyamidino)-pyridyl}]thiophene.

II.C. Prodrugs

In representative embodiments, compounds disclosed herein are prodrugs. A prodrug means a compound that, upon administration to a recipient, is capable of providing (directly or indirectly) a compound of the presently disclosed subject matter or an inhibitorily active metabolite or residue thereof. Prodrugs can increase the bioavailability of the compounds of the presently disclosed subject matter when such compounds are administered to a subject (e.g., by allowing an orally administered compound to be more readily absorbed into the blood) or can enhance delivery of the parent compound to a biological compartment (e.g., the brain or lymphatic system) relative to a metabolite species, for example. A number of the compounds (e.g., Compounds 5, 6, 12, 13, 19, 20, 23, 24, 25, 28, 29, 31, 32, 34, and 35) disclosed herein are prodrugs.

II.D. Pharmaceutically Acceptable Salts

Additionally, the active compounds as described herein can be administered as a pharmaceutically acceptable salt. Such pharmaceutically acceptable salts include the gluconate, lactate, acetate, tartarate, citrate, phosphate, borate, nitrate, sulfate, and hydrochloride salts. The salts of the compounds described herein can be prepared, for example, by reacting the base compound with the desired acid in solution. After the reaction is complete, the salts are crystallized from solution by the addition of an appropriate amount of solvent in which the salt is insoluble. In some embodiments, as described in more detail herein below, the hydrochloride salt of a compound is made by passing hydrogen chloride gas into an ethanolic solution of the free base. In some embodiments, the acetate salt of the presently disclosed diamidine compounds and/or the corresponding N-methoxy analogues are made directly from the appropriate N-hydroxy analogue. In some embodiments, the acetate salt can be made by contacting one of the presently disclosed compounds with a solution comprising acetic acid. Accordingly, in some embodiments, the pharmaceutically acceptable salt is a hydrochloride salt. In some embodiments, the pharmaceutically acceptable salt is an acetate salt.

### III. PHARMACEUTICAL FORMULATIONS

The compounds of Formula (I-III) (including the compounds of Formulas (Ia), (IIa), (IIb), (IIc), (IId), (IIIa), (IIIb), and (IIIc)), the pharmaceutically acceptable salts thereof, prodrugs corresponding to compounds of Formula (I-III), and the pharmaceutically acceptable salts thereof, are all referred to herein as “active compounds.” Pharmaceutical formulations comprising the aforementioned active compounds also are provided herein. These pharmaceutical formulations comprise active compounds as described herein, in a pharmaceutically acceptable carrier. Pharmaceutical formulations can be prepared for oral, intravenous, or aerosol administration as discussed in greater detail below. Also, the presently disclosed subject matter provides such active compounds that have been lyophilized and that can be reconstituted to form pharmaceutically acceptable formulations for administration, for example, as by intravenous or intramuscular injection.

The therapeutically effective dosage of any specific active compound, the use of which is within the scope of embodiments described herein, will vary somewhat from compound to compound, and patient to patient, and will depend upon the condition of the patient and the route of delivery. As a general proposition, a dosage from about 0.1 to about 50 mg/kg will have therapeutic efficacy, with all weights being calculated based upon the weight of the active compound, including the cases where a salt is employed. Toxicity concerns at the higher level can restrict intravenous dosages to a lower level, such as up to about 10 mg/kg, with all weights being calculated based on the weight of the active base, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Preferred dosages are 1 μmol/kg to 50 μmol/kg, and more preferably 22 μmol/kg and 33 μmol/kg of the compound for intravenous or oral administration. The duration of the treatment is usually once per day for a period of two to three weeks or until the condition is essentially controlled. Lower doses given less frequently can be used prophylactically to prevent or reduce the incidence of recurrence of the infection.

In accordance with the present methods, pharmaceutically active compounds as described herein can be administered orally as a solid or as a liquid, or can be administered intramuscularly or intravenously as a solution, suspension, or emulsion. Alternatively, the compounds or salts also can be administered by inhalation, intravenously, or intramuscularly as a liposomal suspension. When administered through inhalation the active compound or salt should be in the form of a plurality of solid particles or droplets having a particle size from about 0.5 to about 5 microns, and preferably from about 1 to about 2 microns.

Pharmaceutical formulations suitable for intravenous or intramuscular injection are further embodiments provided herein. The pharmaceutical formulations comprise a compound of Formula (I-III) described herein, a prodrug as described herein, or a pharmaceutically acceptable salt thereof, in any pharmaceutically acceptable carrier. If a solution is desired, water is the carrier of choice with respect to water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and typically by filtration through a 0.22-micron filter. Subsequent to sterilization, the solution can be dispensed into appropriate receptacles, such as depyrogenated glass vials. The dispensing is preferably done by an aseptic method. Sterilized closures can then be placed on the vials and, if desired, the vial contents can be lyophilized.

In addition to compounds of Formula (I-III) or their salts or prodrugs, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain antimicrobial preservatives. Useful antimicrobial preservatives include methylparaben, propylparaben, and benzyl alcohol. The antimicrobial preservative is typically employed when the formulation is placed in a vial designed for multi-dose use. The pharmaceutical formulations described herein can be lyophilized using techniques well known in the art.

In yet another embodiment of the subject matter described herein, there is provided an injectable, stable, sterile formulation comprising a compound of Formula (I-III), or a salt thereof, in a unit dosage form in a sealed container.

The compound or salt is provided in the form of a lyophilizate, which is capable of being reconstituted with a suitable pharmaceutically acceptable carrier to form a liquid formulation suitable for injection thereof into a subject. The unit dosage form typically comprises from about 10 mg to about 10 grams of the compound salt.

Other pharmaceutical formulations can be prepared from the water-insoluble compounds disclosed herein, or salts thereof, such as aqueous base emulsions. In such an instance, the formulation will contain a sufficient amount of pharmaceutically acceptable emulsifying agent to emulsify the desired amount of the compound or salt thereof. Particularly useful emulsifying agents include phosphatidyl cholines and lecithin.

Additional embodiments provided herein include liposomal formulations of the active compounds disclosed herein. The technology for forming liposomal suspensions is well known in the art. When the compound is an aqueous-soluble salt, using conventional liposome technology, the same can be incorporated into lipid vesicles. In such an instance, due to the water solubility of the active compound, the active compound will be substantially entrained within the hydrophilic center or core of the liposomes. The lipid layer employed can be of any conventional composition and can either contain cholesterol or can be cholesterol-free. When the active compound of interest is water-insoluble, again employing conventional liposome formation technology, the salt can be substantially entrained within the hydrophobic lipid bilayer that forms the structure of the liposome. In either instance, the liposomes that are produced can be reduced in size, as through the use of standard sonication and homogenization techniques.

The liposomal formulations comprising the active compounds disclosed herein can be lyophilized to produce a lyophilizate, which can be reconstituted with a pharmaceutically acceptable carrier, such as water, to regenerate a liposomal suspension.

Pharmaceutical formulations also are provided which are suitable for administration as an aerosol by inhalation. These formulations comprise a solution or suspension of a desired compound described herein or a salt thereof, or a plurality of solid particles of the compound or salt. The desired formulation can be placed in a small chamber and nebulized. Nebulization can be accomplished by compressed air or by ultrasonic energy to form a plurality of liquid droplets or solid particles comprising the compounds or salts. The liquid droplets or solid particles should have a particle size in the range of about 0.5 to about 10 microns, more preferably from about 0.5 to about 5 microns. The solid particles can be obtained by processing the solid compound or a salt thereof, in any appropriate manner known in the art, such as by micronization. Most preferably, the size of the solid particles or droplets will be from about 1 to about 2 microns. In this respect, commercial nebulizers are available to achieve this purpose. The compounds can be administered via an aerosol suspension of respirable particles in a manner set forth in U.S. Pat. No. 5,628,984, the disclosure of which is incorporated herein by reference in its entirety.

When the pharmaceutical formulation suitable for administration as an aerosol is in the form of a liquid, the formulation will comprise a water-soluble active compound in a carrier that comprises water. A surfactant can be present, which lowers the surface tension of the formulation sufficiently to result in the formation of droplets within the desired size range when subjected to nebulization.

As indicated, both water-soluble and water-insoluble active compounds are provided. As used herein, the term “water-soluble” is meant to define any composition that is soluble in water in an amount of about 50 mg/mL, or greater. Also, as used herein, the term “water-insoluble” is meant to define any composition that has a solubility in water of less than about 20 mg/mL. In some embodiments, water-soluble compounds or salts can be desirable whereas in other embodiments water-insoluble compounds or salts likewise can be desirable.

### IV. METHODS FOR TREATING MICROBIAL INFECTIONS

Subjects with microbial infections can be treated by methods described herein. Such infections can be caused by a variety of microbes, including fungi, algae, protozoa, bacteria, and viruses. Exemplary microbial infections that can be treated by the method of the presently disclosed subject matter include, but are not limited to, infections caused by Trypanosoma spp. (e.g., Trypanosoma brucei rhodesiense, Trypanosoma brucei gambiense, Trypanosoma brucei brucei, and Trypanosoma cruzi), Plasmodium spp. (e.g., Plasmodium falciparum), Mycobacterium tuberculosis, Pneumocystis carinii, Giardia lamblia, Cryptosporidium parvum, Cryptococcus neoformans, Candida albicans, Candida tropicalis, Salmonella typhimurium, Leishmania donovani, and Leishmania mexicana amazonensis. As used herein the terms Trypanosoma spp., Plasmodium spp., and Leishmania spp. encompass microbes classified under the genera Trypanosoma, Plasmodium, and Leishmania respectively.

The methods of the presently disclosed subject matter are useful for treating these conditions in that they inhibit the onset, growth, or spread of the condition, cause regression of the condition, cure the condition, or otherwise improve the general well-being of a subject afflicted with, or at risk of, contracting the condition. Thus, in accordance with the presently disclosed subject matter, the terms “treat,” “treating,” and grammatical variations thereof, as well as the phrase “method of treating,” are meant to encompass any desired therapeutic intervention, including but not limited to a method for treating an existing infection in a subject, and a method for the prophylaxis (i.e., preventing) of infection, such as in a subject that has been exposed to a microbe as disclosed herein or that has an expectation of being exposed to a microbe as disclosed herein.

The methods for treating microbial infections comprise administering to a subject in need thereof an active compound as described herein. These active compounds, as set forth above, include compounds of Formula (I-III) (including the compounds of Formulas (Ia), (IIa), (IIb), (IIc), (IId), (IIIa), (IIIb), and (IIIc)), their corresponding prodrugs, and pharmaceutically acceptable salts of the compounds and prodrugs. In some embodiments, the compound of Formula (I-III) is administered to a subject with an existing microbial infection. In some embodiments, the compound of Formula (I-III) is administered prophylactically to prevent a microbial infection or to prevent the recurrence of a microbial infection. Thus, in some embodiments, the compound of Formula (I-III) is administered prophylactically to prevent or reduce the incidence of one of: (a) a microbial infection in a subject at risk of infection; (b) a recurrence of the microbial infection; and (c) combinations thereof.

The subject treated in the presently disclosed subject matter in its many embodiments is desirably a human subject, although it is to be understood the methods described herein are effective with respect to all vertebrate species, which are intended to be included in the term “subject.” The methods described herein are particularly useful in the treatment and/or prevention of infectious diseases in warm-blooded vertebrates. Thus, the methods can be used as treatment for mammals and birds.

More particularly, provided herein is the treatment of mammals, such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided herein is the treatment of birds, including the treatment of those kinds of birds that are endangered, kept in zoos or as pets (e.g., parrots), as well as fowl, and more particularly domesticated fowl, i.e., poultry, such as turkeys, chickens, ducks, geese, guinea fowl, and the like, as they also are of economical importance to humans. Thus, embodiments of the methods described herein include the treatment of livestock, including, but not limited to, domesticated swine (pigs and hogs), ruminants, horses, poultry, and the like.

With regard to the presently described method embodiments, compounds of Formula (I) can be defined as having a structure as follows:

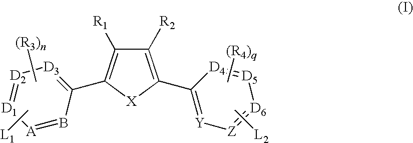

wherein:

X is Se or S;

D₁, D₂, D₃, D₄, D₅, and D₆ are independently C or N;

A, B, Y, and Z are each independently selected from the group consisting of CH and N, provided that when X is S, at least two of A, B, Y, and Z are N;

n is an integer from 0 to 2;

q is an integer from 0 to 2;

R₁ and R₂ are independently selected from the group consisting of H, alkyl, substituted alkyl, cycloalkyl, aryl, substituted aryl, aralkyl, halogen, alkoxyl, aryloxyl, aralkoxyl, and hydroxyl;

each R₃ and R₄ is independently selected from the group consisting of H, alkyl, substituted alkyl, cycloalkyl, aryl, substituted aryl, aralkyl, halogen, alkoxyl, aryloxyl, aralkoxyl, and hydroxyl; and

L₁ and L₂ are independently selected from the group consisting of:

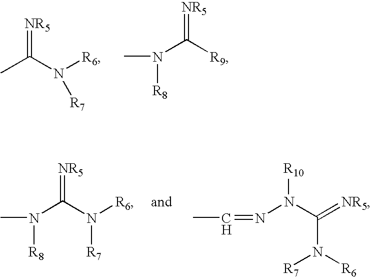

wherein:

each R₅ is selected from the group consisting of H, hydroxyl, alkyl, cycloalkyl, aryl, aralkyl, alkoxyl, aryloxyl, hydroxycycloalkyl, alkoxycycloalkyl, hydroxyalkyl, aminoalkyl, acyloxyl, and alkylaminoalkyl; and

each R₆, R₇, R₈, R₉, and R₁₀ is independently selected from the group consisting of H, alkyl, alkoxyalkyl, cycloalkyl, aryl, aralkyl, hydroxyalkyl, aminoalkyl, and alkylaminoalkyl, or a R₅ and a R₆ or a R₅ and a R₉ together represent a C₂ to C₁₀ alkylene, or a R₅ and a R₆ or a R₅ and a R₉ together are:

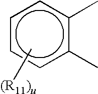

wherein u is an integer from 1 to 4, and R₁₁ is H or —CONHR₁₂NR₁₃R₁₄, wherein R₁₂ is alkylene and R₁₃ and R₁₄ are each independently selected from the group consisting of H, alkyl, aryl and aralkyl; or

a pharmaceutically acceptable salt thereof.

In some embodiments, each of D₁, D₂, D₃, D₄, D₅, and D₆ is carbon (i.e., C) and the compound of Formula (I) is a compound of Formula (Ia):

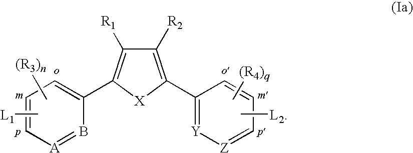

In some embodiments, L₁ is attached at the p carbon and L₂ is attached at the p′ carbon.

In some embodiments, X is Se and the compound of Formula (I) is a diaryl selenophene compound having a structure of Formula (II):

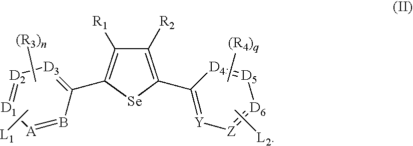

In some embodiments, each of D₁, D₂, D₃, D₄, D₅, and D₆ is C and the compound of Formula (II) has a structure of Formula (IIa):

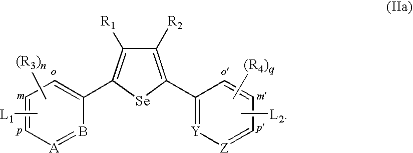

In some embodiments, L₁ is attached at the p carbon and L₂ is attached at the p′ carbon and the compound of Formula (IIa) has a structure of Formula (IIb):

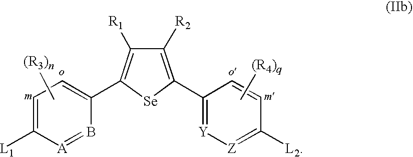

In some embodiments, each of A, B, Y, and Z of the compound of Formula (IIb) is CH. In some embodiments, at least one of A, B, Y, and Z of the compound of Formula (IIb) is N. In some embodiments, B and Y are each N.

In some embodiments, the compound of Formula (II) has a structure of Formula (IIc):

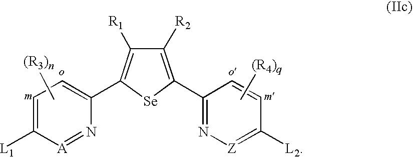

In some embodiments, A and Z are each N. In some embodiments, the compound of Formula (II) has a structure of Formula (IId):

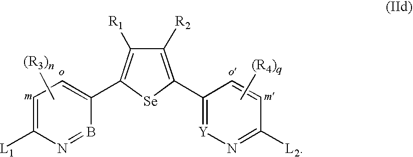

In some embodiments, X is S and the compound of Formula (I) has a structure of Formula (III):

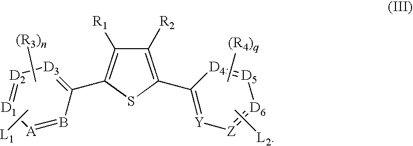

wherein at least two of A, B, Y and Z are N. In some embodiments, each of D₁, D₂, D₃, D₄, D₅, and D₆ is C and the compound of Formula (III) has a structure of Formula (IIIa):

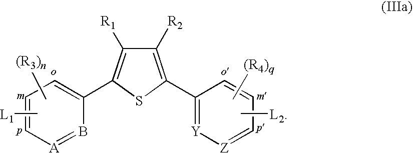

In some embodiments, A and Z are both N or B and Y are both N, and the compound of Formula (III) has a structure of one of Formulas (IIIb) and (IIIc):

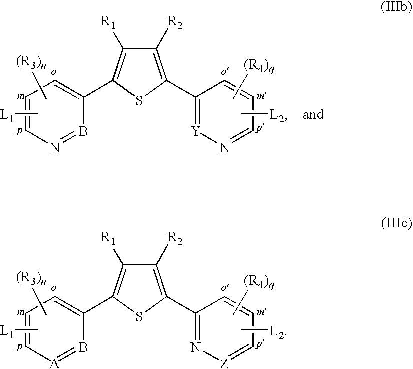

In some embodiments, the presently disclosed method comprises administering one or more of the following compounds to a subject in need of treatment thereof: 2,5-Bis(4-amidinophenyl)selenophene; 2,5-Bis[4-(N-hydroxyamidino)phenyl]selenophene; 2,5-Bis[4-(N-methoxyamidino)phenyl]-selenophene; 6-[5-(4-amidinophenyl)-selenophen-2-yl]nicotinamidine; 6-{5-[4-(N-hydroxyamidino)phenyl]selenophen-2-yl}-N-hydroxynicotinamidine; 6-{5-[4-(N-methoxyamidino)phenyl]selenophen-2-yl}-N-methoxynicotinamidine; 5-[5-(4-amidinophenyl)selenophene-2-yl]-pyridine-2-amidine; 5-{5-[4-(N-hydroxy-amidino)phenyl]selenophen-2-yl}-pyridine-2-N-hydroxamidine; 5-{5-[4-(N-methoxyamidino)phenyl]selenophen-2-yl}pyridine-2-N-methox-amidine; 2,5-Bis-[5-(2-amidinopyridyl)]selenophene; 2,5-Bis-[5-{2-(N-hydroxyamidino)pyridyl}]-selenophene; 2,5-Bis-[5-{2-(N-methoxyamidino)pyridyl}]selenophene; 2,5-Bis-[5-{2-N—(N,N-dimethylaminoacetoxyamidino)pyridyl}]selenophene; 2,5-Bis-[2-(5-amidinopyridyl)]selenophene; 2,5-Bis-[2-{5-(N-hydroxyamidino)pyridyl}]-selenophene; 2,5-Bis-[2-{5-(N-methoxyamidino)pyridyl}]selenophene; 2,5-Bis-[2-(5-amidinopyridyl)]thiophene; 2,5-Bis-[2-{5-(N-hydroxyamidino)-pyridyl}]thiophene; 2,5-Bis-[2-{5-(N-methoxyamidino)-pyridyl}]thiophene; 2,5-Bis-[5-(2-amidinopyridyl)]thiophene; 2,5-Bis-[5-{2-(N-hydroxyamidino)pyridyl}]-thiophene; and 2,5-Bis-[5-{2-(N-methoxyamidino)pyridyl}]thiophene.

In some embodiments, one of more of the compounds of Formula (I-III) are administered to a subject in the form of a pharmaceutically acceptable salt. In some embodiments, the pharmaceutically acceptable salt is a hydrochloride salt.

The compound of Formula (I-III) can be administered in any convenient method: orally, intravenously, subcutaneously, intramuscularly, etc. In some embodiments, the compound can be administered in an aerosol formulation. In some embodiments, the compound of Formula (I-III) is a prodrug and the compound is administered orally.

In some embodiments, the microbial infection is a protozoal infection. In some embodiments, the microbial infection is an infection of a Trypanosoma species. In some embodiments, the microbial infection is selected from the group consisting of a Trypanosoma brucei rhodesiense infection, a Plasmodium falciparum infection, and a Leishmania donovoni infection.

In some embodiments, the microbial infection is a Trypanosoma brucei rhodesiense infection and the compound is selected from the group consisting of 2,5-Bis(4-amidinophenyl)selenophene; 6-[5-(4-amidinophenyl)selenophen-2-yl]nicotinamidine; 5-[5-(4-amidinophenyl)selenophene-2-yl]pyridine-2-amidine; 5-{5-[4-(N-methoxyamidino)phenyl]selenophen-2-yl}-pyridine-2-N-methoxy-amidine; 2,5-Bis-[5-(2-amidinopyridyl)]selenophene; 2,5-Bis-[2-(5-amidinopyridyl)]selenophene; 2,5-Bis-[2-(5-amidinopyridyl)]thiophene; and 2,5-Bis-[5-(2-amidinopyridyl)]thiophene.

In some embodiments, the microbial infection is a Plasmodium falciparum infection and the compound is selected from the group consisting of 2,5-Bis(4-amidinophenyl)selenophene; 6-[5-(4-amidinophenyl)selenophen-2-yl]-nicotinamidine; 5-[5-(4-amidinophenyl)selenophene-2-yl]-pyridine-2-amidine; 2,5-Bis-[5-(2-amidinopyridyl)]selenophene; 2,5-Bis-[2-(5-amidinopyridyl)]-selenophene; and 2,5-Bis-[5-(2-amidinopyridyl)]thiophene.

In some embodiments, the microbial infection is a Leishmania donovani infection and the compound is selected from the group consisting of 2,5-Bis(4-amidinophenyl)selenophene; 6-[5-(4-amidinophenyl)-selenophen-2-yl]-nicotinamidine; 5-[5-(4-amidinophenyl)selenophene-2-yl]pyridine-2-amidine; 2,5-Bis-[5-(2-amidinopyridyl)]selenophene; and 2,5-Bis-[5-(2-amidinopyridyl)]-thiophene.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following

Examples are intended to be exemplary only and that numerous changes, modifications, and alterations can be employed without departing from the scope of the presently disclosed subject matter.

### Methods and Materials

Melting points were recorded using a Thomas-Hoover (Uni-Melt) capillary melting point apparatus (Thomas Scientific, Swedesboro, N.J., United States of America) and are uncorrected. TLC analysis was carried out on silica gel 60 F₂₅₄ precoated aluminum sheets and detected under UV light. ¹H and ¹³C NMR spectra were recorded employing a Varian GX400 or Varian Unity Plus 300 spectrometer (Varian, Inc., Palo Alto, Calif., United States of America), and chemical shifts (δ) are in ppm relative to TMS as internal standard. Mass spectra were recorded on a VG analytical 70-SE spectrometer (VG Analytical, Ltd., Manchester, United Kingdom). Elemental analyses were obtained from Atlantic Microlab Inc. (Norcross, Ga., United States of America) and are within ±0.4 of the theoretical values. The compounds reported as salts frequently analyzed correctly for fractional moles of water and/or ethanol of solvation. In each case, proton NMR showed the presence of indicated solvent(s). Unless otherwise indicated, chemical reagents and solvents were obtained from Aldrich Chemical Co. (St. Louis, Mo., United States of America), Fisher Scientific (Fairlawn, N.J., United States of America), Frontier Scientific (Logan, Utah, United States of America) or Lancaster Synthesis, Inc. (Windham, N.H., United States of America).

### Example 1

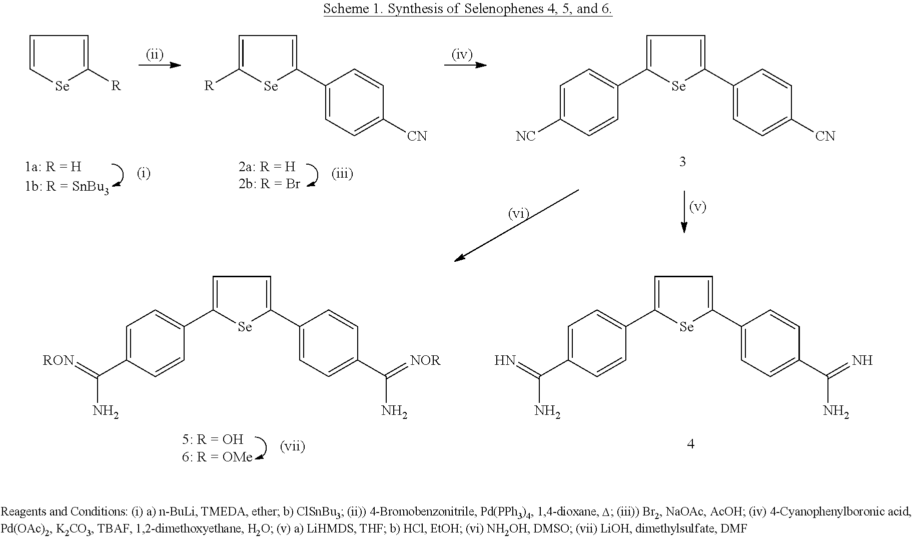

2-(4-Cyanophenyl)selenophene (2a). Referring now to Scheme 1 above, 1b (i.e., 2-(tributylstannyl)selenophene, prepared from 1a (i.e., selenophene) as previously described in Heterocycles, 30, 651, (1990); 8.82 g; 21.0 mmol) was added to a solution of 4-bromobenzonitrile (3.73 g, 20.5 mmol) and catalytic Pd(PPh₃)₄ (280 mg) in 1,4-dioxane (60 mL). The mixture was heated under nitrogen at 100° C. for approximately 18 h. After the dark mixture was cooled, silica gel was added and the solvent was removed in vacuo. The resulting solid was then chromatographed over silica gel eluting with a gradient of 100% hexanes to 5% EtOAc in hexanes. The homogeneous fractions were combined and concentrated in vacuo to give a yellow solid (2.93 g, 60%), which was recrystallized from hexanes to give yellow powdery crystals (2.35 g, 48%), mp 80.5-81° C. ¹H-NMR (CDCl₃): 7.36 (dd, J=5.4 and 3.9 Hz, 1H), 7.58 (dd, J=3.9 and 0.9 Hz, 1H), 7.64 (s, 4H), 8.07 (dd, J=5.7 and 1.2 Hz, 1H). IR (cm⁻¹): 3098, 2226, 1608, 1600, 1494, 1436, 1413, 824, 701, 564, 547. Anal. Calcd. for C₁₁H₇NSe (232.14): C, 56.91; H, 3.04; N, 6.03. Found: C, 56.90; H, 2.93; N, 5.96.
2-Bromo-5-(4-cyanophenyl)selenophene (2b). To a chilled solution of 2a (2.40 g, 10.34 mmol) and NaOAc (0.86 g, 10.40 mmol) in AcOH (60 mL) was added, in portions, a solution of bromine (1.70 g, 10.6 mmol) in AcOH (5 mL) over a course of approximately 10 min. The resulting suspension was stirred for an additional 15 min at room temperature and then diluted with water (75 mL) to give a thick precipitate which was filtered and rinsed with water. Recrystallization from MeOH (approximately 100 mL)/water (approximately 20 mL) gave off-white solid 2b (2.77 g, 86%), mp 115-116.5° C. ¹H-NMR (DMSO-d₆): 7.49 (d, J=3.9 Hz, 1H), 7.67 (d, J=4.2 Hz, 1H), 7.76 (d, J=8.4 Hz, 2H), 7.84 (d, J=8.4 Hz, 2H). IR (cm⁻¹): 3071, 2230, 1605, 1497, 1437, 1409, 832, 803, 537, 489. Anal. Calcd. for C₁₁H₆BrNSe (311.04): C, 42.48; H, 1.94; N, 4.50. Found: C, 42.44; H, 1.72; N, 4.46.
2,5-Bis(4-cyanophenyl)selenophene (3). To a mixture of 2b (0.622 g, 2.0 mmol), 4-cyanophenylboronic acid (0.32 g, 2.18 mmol), catalytic palladium(II) acetate (12 mg), and tetrabutylammonium fluoride (TBAF hydrate) (0.62 g, 2.4 mmol) in 1,2-dimethoxyethane (DME) (20 mL) was added a solution of K₂CO₃ (0.56 g, 4.0 mmol) in water (4 mL). The entire resulting mixture was heated under nitrogen at 90° C. with good stirring for 2 h. After standing at room-temperature for 30 min, the precipitated product was then filtered and rinsed with water and then with diethyl ether to give a green solid. The green solid was recrystallized from an acetone/EtOH mixture (with partial concentration to remove the acetone) to give a mustard yellow solid (0.42 g, 63%), mp 240-249° C. A second recrystallization from n-BuOH gave fluffy yellow micro-needles (0.36 g, 54%), mp 248-250° C. ¹H-NMR (DMSO-d₆): 7.87 (s, 8H), 7.97 (s, 2H). IR (cm⁻¹): 3082, 3050, 2224, 1600, 1556, 1494, 1458, 1411, 1288, 1175, 1123, 836, 803, 469, 452. Anal. Calcd. for C₁₈H₁₀N₂Se (333.25): C, 64.87; H, 3.02; N, 8.41. Found: C, 64.42; H, 2.95; N, 8.23.
2,5-Bis(4-amidinophenyl)selenophene Dihydrochloride (4). To a suspension of 3 (0.145 g, 0.435 mmol) in dry THF (4 mL) was added lithium bis(trimethylsilyl)amide (LiHMDS) (2.0 mL of a 1.06 M solution, 2.12 mmol). After stirring for 30 min, a clear yellow-orange solution was achieved. After continued stirring overnight, the solution was chilled and treated with HCl-saturated EtOH (approximately 2 mL) to give a yellow-orange precipitate. After stirring an additional 24 h, the suspension was concentrated in vacuo to near dryness, diluted with a small volume of water and filtered to give, after rinsing with a small volume of water and drying in vacuo, an orange solid (0.13 g), mp>370° C. ¹H-NMR (DMSO-d₆): 7.91 (s, 8H), 8.01 (s, 2H), 9.32 (br s, 8H). MS (ESI): Calcd. for C₁₈H₁₆N₄⁸⁰Se: 368. Found: 368.1 (100%). Anal. Calcd. for C₁₈H₁₆N₄Se.2HCl (440.22): C, 49.11; H, 4.12; N, 12.73. Found: C, 48.85; H, 4.16; N, 12.59.
2,5-Bis[4-(N-hydroxyamidino)phenyl]selenophene Dihydrochloride (5). Dinitrile 3 (0.72 g, 2.16 mmol) was converted to the free base of the title compound by reaction with 8 molar equivalents of hydroxylamine (generated by reaction of hydroxylamine hydrochloride with potassium t-butoxide in approximately 50 mL DMSO). After stirring the mixture overnight at room temperature, the suspension was heated at about 40° C. for 3-4 h to give an orange-yellow solution. This solution was then diluted with excess water to give a yellow solid, which was filtered off, rinsed with water and air dried. Yield: 0.83 g (96%). ¹H-NMR (DMSO-d₆): 5.85 (br s, 4H), 7.63 (d, J=8.7 Hz, 4H), 7.71 (d, J=8.7 Hz, 4H), 7.74 (s, 2H), 9.72 (s, 2H). To give the salt, the free base was dissolved in approximately 400 mL hot EtOH that had been treated with dry HCl, gravity filtered to remove trace insolubles, and then concentrated to an orange solid. ¹H-NMR (DMSO-d₆): 7.78 (d, J=8.7 Hz, 4H), 7.88 (d, J=8.4 Hz, 4H), 7.97 (s, 2H), 8.92 (br s, 4H), 11.38 (br s, 2H). Anal. Calcd. for C₁₈H₁₆N₄O₂Se.2HCl.0.75H₂O (485.74): C, 44.51; H, 4.05; N, 11.53. Found: C, 44.69; H, 3.99; N, 11.14.
2,5-Bis[4-(N-methoxyamidino)phenyl]selenophene Dihydrochloride (6). To a solution of 2,5-bis[4-(N-hydroxyamidino)phenyl]selenophene (free base of 5; 0.60 g, 1.50 mmol) in DMF (40 mL) was added a solution of LiOH hydrate (0.25 g, 6.0 mmol) in water (2.5 mL). The resulting thick suspension was then treated with dimethylsulfate (0.47 g, 3.73 mmol) and stirred vigorously at room temperature for 3 h. The resulting orange solution was then poured into water (150 mL) to give a yellow solid, which was filtered off, rinsed with water and air dried (yield: 0.57 g). The solid was recrystallized from EtOH (about 400 mL) to give a yellow crystalline solid (0.35 g, 55%), mp>270° C. The free base was then suspended in EtOH and treated with anhydrous HCl. After stirring the suspension overnight, it was concentrated to a low volume, diluted with ethyl ether and filtered to give the dihydrochloride salt (0.40 g, 98%) as a yellow solid. ¹H-NMR (DMSO-d₆): 3.83 (s, 6H), 7.79 (s, 8H), 7.90 (s, 2H). MS (ESI): Calcd. for C₂₀H₂₀N₄O₂⁸⁰Se: 428. Found: 429 (MH⁺, 100%). Anal. Calcd. for C₂₀H₂₀N₄O₂Se.2HCl (500.28): C, 48.02; H, 4.43; N, 11.20. Found: C, 47.99; H, 4.48; N, 10.83.

### Example 2

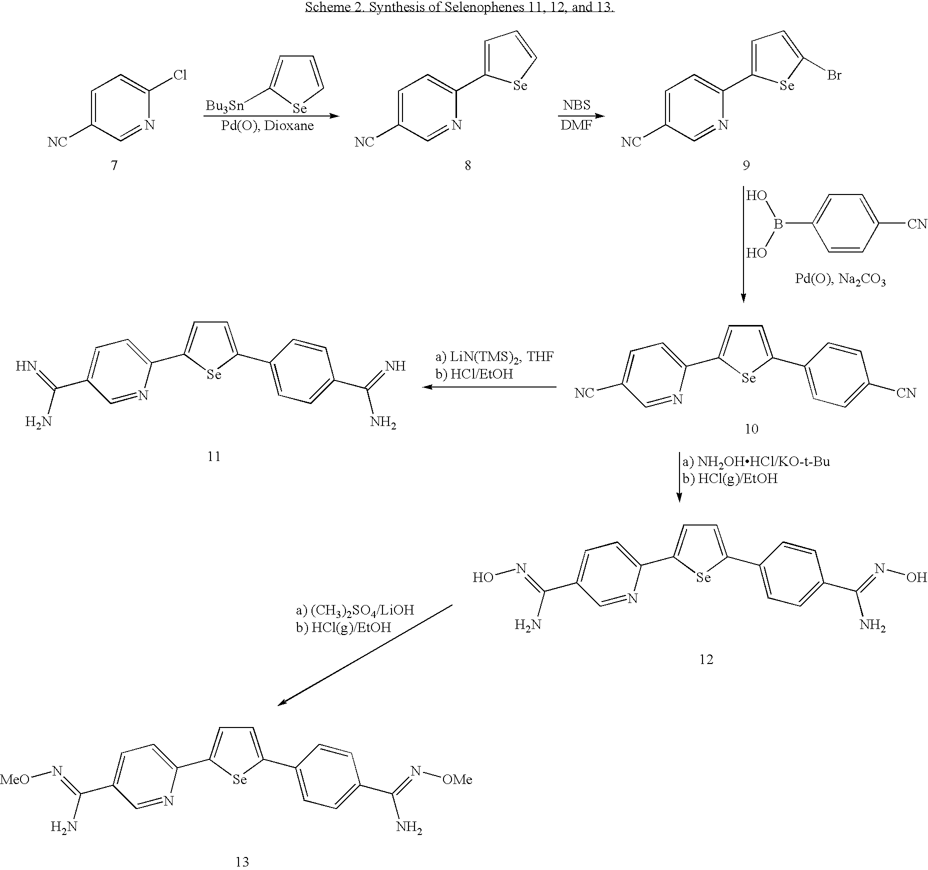

6-(Selenophen-2-yl)nicotinonitrile (8). Referring now to Scheme 2 above, a mixture of 7 (i.e., 6-chloronicotinonitrile, 4.155 g, 30 mmol), 2-tributylstannylselenophene (30 mmol), and tetrakis(triphenylphosphine)palladium (500 mg) in dry dioxane (60 mL) was heated under nitrogen at reflux (100-110° C.) for 24 h. The solvent was evaporated under reduced pressure. The solid was dissolved in ethyl acetate and the resulting solution was passed through celite to remove Pd. The solvent was evaporated, and the solid was filtered to give 8 in 83% yield; mp 108-108.5° C. ¹H NMR (DMSO-d₆): 7.45 (dd, J=5.4, 3.9 Hz, 1H), 8.11 (d, J=8.4 Hz, 1H), 8.17 (d, J=3.9 Hz, 1H), 8.28 (dd, J=8.4, 2.4 Hz, 1H), 8.40 (d, J=5.4 Hz, 1H), 8.90 (d, J=2.4 Hz, 1H). ¹³C NMR (DMSO-d₆): 156.2, 152.7, 149.3, 140.6, 137.3, 131.6, 130.2, 117.6, 117.3, 106.5. MS (ESI) m/e (rel. int.); 234 (M⁺, 100), 208 (5), 154 (20), 142 (35). Anal. Calcd. for C₁₀H₆N₂Se: C, 51.52; H, 2.59. Found: C, 51.86; H, 2.71.
6-(5-Bromo-selenophen-2-yl)-nicotinonitrile (9). To a solution of 8 (4.68 g, 20 mmol) in DMF (20 mL) was added portionwise N-bromosuccinimide (22 mmol) with stirring. The reaction mixture was stirred overnight at room temperature, then poured onto cold water. The precipitate which formed was collected, washed with water and dried to give 9 in 95% yield; mp 182.5-184° C. (DMF). ¹H NMR (DMSO-d₆): 7.53 (d, J=4.2 Hz, 1H), 7.92 (d, J=4.2 Hz, 1H), 8.06 (d, J=7.8 Hz, 1H), 8.27 (d, J=7.8 Hz, 1H), 8.86 (s, 1H). ¹³C NMR (DMSO-d₆): 155.2, 152.8, 151.2, 140.9, 135.8, 130.5, 120.7, 117.4, 117.2, 106.9. MS (ESI) m/e (rel. int.); 312 (M⁺, 80), 233 (100), 206 (5), 153 (90). Anal. Calcd. for C₁₀H₅BrN₂Se: C, 38.49; H, 1.62. Found: C, 38.13; H, 1.55.
6-[5-(4-Cyanophenyl)-selenophen-2-yl]-nicotinonitrile (10). To a stirred solution of 9 (1.56 g, 5 mmol), and tetrakis(triphenylphosphine)palladium (288 mg) in toluene (10 mL) under a nitrogen atmosphere was added 5 mL of a 2 M aqueous solution of Na₂CO₃ followed by 4-cyanophenylboronic acid (876 mg, 6 mmol) in 5 mL of methanol. The vigorously stirred mixture was warmed to 80° C. for 24 h, then cooled, and the precipitate was filtered. The precipitate was partitioned between methylene chloride (500 mL) and 2 M aqueous Na₂CO₃ (25 mL) containing 3 mL of concentrated ammonia. The organic layer was dried over Na₂SO₄, filtered, and then concentrated to dryness under reduced pressure to afford 10 in 78% yield; mp 289.5-291° C. (DMF). ¹H NMR (DMSO-d₆): 7.80-7.95 (m, 5H), 8.11-8.29 (m, 3H), 8.91 (s, 1H). ¹³C NMR (DMSO-d₆): 155.3, 152.3, 151.3, 150.1, 140.2, 139.3, 132.6, 130.9, 129.7, 126.2, 118.1, 117.5, 116.7, 110.2, 106.7. MS (ESI) m/e (rel. int.); 335 (M⁺, 100), 254 (40). Anal. Calcd. for C₁₇H₉N₃Se: C, 61.09; H, 2.71. Found: C, 60.93; H, 2.72.
6-[5-(4-Amidinophenyl)-selenophen-2-yl]-nicotinamidine (11). Dinitrile 10 (334 mg, 1 mmol), suspended in freshly distilled THF (5 mL), was treated with lithium trimethylsilylamide (1M solution in THF, 6 mL, 6 mmol) and the reaction was allowed to stir overnight. The reaction mixture was then cooled to 0° C. and HCl saturated ethanol (70 mL) was added, whereupon precipitate started forming. The mixture was left overnight and diluted with ether. The solid was collected by filtration. The diamidine was purified by neutralization with 1N NaOH followed by filtration of the resultant solid and washing with water (3 times). Finally, the free base was stirred with ethanolic HCl overnight, diluted with ether, and the solid which formed was filtered and dried to give the hydrochloride salt of diamidine 11 in 87% yield; mp>300° C. ¹H NMR (D₂O/DMSO-d₆): 7.84-7.91 (m, 5H), 8.07-8.21 (m, 3H), 8.86 (s, 1H). ¹³C NMR (D₂O/DMSO-d₆): 165.2, 163.7, 156.9, 151.6, 150.5, 149.1, 140.6, 137.2, 131.2, 130.1, 129.2, 127.1, 126.4, 122.8, 117.7. MS (ESI) m/e (rel. int.); 369 (M⁺, 20), 370 (M⁺+1, 40), 224 (10), 185 (100). Anal. Calcd. for C₁₇H₁₅N₅Se.2.6HCl.0.25C₂H₅OH: C, 44.29; H, 4.02; N, 14.75. Found: C, 44.58; H, 3.89; N, 14.48.
6-{5-[4-(N-hydroxyamidinophenyl]-selenophen-2-yl}-N-hydroxynicotin-amidine (12). A mixture of hydroxylamine hydrochloride (2.08 g, 30 mmol, 10 eq.) in anhydrous DMSO (20 mL) was cooled to 5° C. under nitrogen and potassium t-butoxide (3.36 g, 30 mmol, 10 eq.) was added in portions. The mixture was stirred for 30 min. The mixture was then added to dinitrile 10 (3 mmol, 1 eq.). This reaction mixture was stirred overnight at room temperature. The reaction mixture was then poured slowly onto ice water. The precipitate was filtered and washed with water and then ethanol to afford 12 in 98% yield; mp 231-232.5° C. ¹H NMR (DMSO-d₆): 5.87 (s, 2H), 5.97 (s, 2H), 7.63 (d, J=8.4 Hz, 2H), 7.68 (d, J=8.4 Hz, 2H), 7.75 (d, J=3.9 Hz, 1H), 7.91-8.02 (m, 3H), 8.74 (d, J=2.1 Hz, 1H), 9.72 (s, 1H), 9.86 (s, 1H). ¹³C NMR (DMSO-d₆): 153.0, 150.8, 150.4, 149.9, 148.7, 146.5, 136.0, 133.6, 132.6, 128.7, 127.7, 127.4, 126.0, 125.4, 117.0. MS (ESI) m/e (rel. int.); 402 (M⁺+1, 40), 386 (80), 370 (30), 353 (15), 265 (100). Hydrochloride salt of 12: mp>300° C. Anal. Calcd. for C₁₇H₁₅N₅O₂Se.3.0HCl.1.75H₂O: C, 37.72; H, 3.98; N, 12.90. Found: C, 37.65; H, 3.64; N, 12.65.
6-{5-[4-(N-Methoxyamidino)-phenyl]-selenophen-2-yl}-N-methoxynicotin-amidine (13). To a suspension of diamidoxime 12 (442 mg, 1.1 mmol) in DMF (15 mL) was added LiOH.H₂O (277 mg, 6.6 mmol, in 4 mL H₂O) and then dimethylsulfate (690 mg, 5.5 mmol). The reaction mixture was kept stirring overnight, after which it was poured onto ice water, and the precipitate was filtered, washed with water, and dried to give 13 in 80% yield; mp 172-173° C. (DMF). ¹H NMR (DMSO-d₆): 3.75 (s, 3H), 3.77 (s, 3H), 6.10 (s, 2H), 6.25 (s, 2H), 7.69 (s, 4H), 7.79 (d, J=4.2 Hz, 1H), 7.98-8.03 (m, 3H), 8.75 (s, 1H). ¹³C NMR (DMSO-d₆): 153.4, 150.9, 150.5, 149.9, 148.9, 146.7, 136.3, 134.0, 131.9, 128.9, 127.8, 126.6, 126.4, 125.4, 117.0, 60.7, 60.6. MS (ESI) m/e (rel. int.); 429 (M⁺, 100), 398 (25), 382 (25), 351 (50). Hydrochloride salt of 13: mp 218-219° C. Anal. Calcd. for C₁₉H₁₉N₅O₂Se.3.0HCl.0.7H₂O.0.5C₂H₅OH: C, 41.90; H, 4.64; N, 12.19. Found: C, 42.19; H, 4.50; N, 11.85.

### Example 3

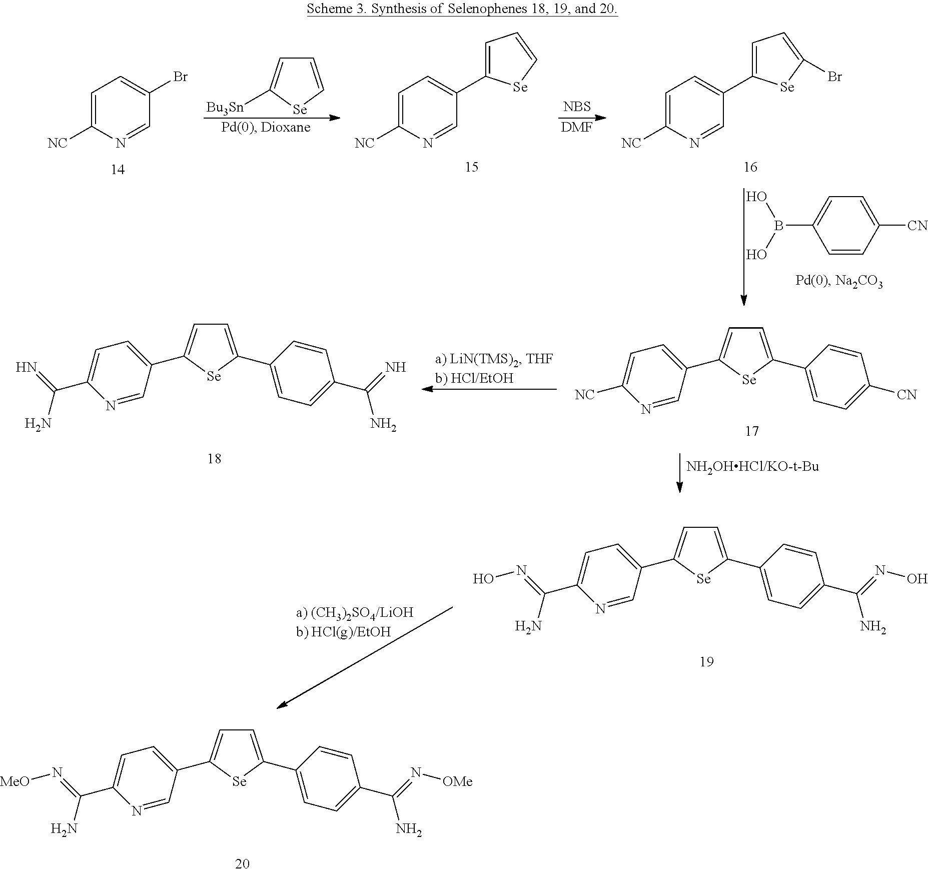

5-(Selenophen-2-yl)pyridine-2-carbonitrile (15). Referring now to Scheme 3 above, compound 15 was prepared in 76% yield from 5-bromo-pyridine-2-carbonitrile (14) using a Stille coupling analogous to that used to prepare compound 8 in Example 2. Compound 15: mp 100-101° C. ¹H NMR (DMSO-d₆): 7.43 (dd, J=5.4, 3.9 Hz, 1H), 7.94-7.98 (m, 2H), 8.19 (dd, J=8.1, 2.4 Hz, 1H), 8.38 (d, J=5.4 Hz, 1H), 8.99 (d, J=2.4 Hz, 1H). ¹³C NMR (DMSO-d₆): 147.8, 143.7, 135.6, 135.1, 133.7, 131.2, 130.4, 129.8, 129.2, 117.6. MS (ESI) m/e (rel. int.); 234 (M⁺, 100), 207 (5), 154 (50). Anal. Calcd. for C₁₀H₆N₂Se: C, 51.52; H, 2.59. Found: C, 51.69; H, 2.55.
5-(5-Bromo-selenophen-2-yl)pyridine-2-carbonitrile (16). Using the same procedure described for 9 above, except starting with 15, compound 16 was prepared in 93% yield; mp 158-159° C. (DMF). ¹H NMR (DMSO-d₆): 7.54 (d, J=4.2 Hz, 1H), 7.80 (d, J=4.2 Hz, 1H), 8.04 (d, J=8.1 Hz, 1H), 8.20 (dd, J=8.1, 2.4 Hz, 1H), 8.99 (d, J=2.4 Hz, 1H). ¹³C NMR (DMSO-d₆): 147.7, 145.8, 135.1, 134.2, 133.7, 130.7, 130.2, 129.2, 118.3, 117.4. MS (ESI) m/e (rel. int.); 312 (M⁺, 100), 233 (25), 206 (15), 141 (40). Anal. Calcd. for C₁₀H₅BrN₂Se: C, 38.49; H, 1.62. Found: C, 38.25; H, 1.67.
5-[5-(4-Cyanophenyl)-selenophen-2-yl]-pyridine-2-carbonitrile (17). Adopting the Suzuki coupling procedure described above in Example 2 for compound 10, starting with selenophene 16 and using NaHCO₃ as a base, dinitrile 17 was produced in 71% yield; mp 266.5-268° C. (DMF). ¹H NMR (DMSO-d₆): 7.80-7.85 (m, 4H), 7.91-8.02 (m, 3H), 8.22 (dd, J=8.1, 2.4 Hz, 1H), 9.03 (d, J=2.4 Hz, 1H). ¹³C NMR (DMSO-d₆): 149.9, 147.4, 144.6, 138.9, 134.1, 133.4, 132.6, 130.8, 130.6, 129.4, 128.7, 126.1, 118.0, 116.9, 110.1. MS (ESI) m/e (rel. int.); 335 (M⁺, 100), 254 (25).
5-[5-(4-Amidinophenyl)-selenophen-2-yl]-pyridine-2-amidine (18). The same procedure described for 11 was followed, except starting with dinitrile 17, to furnish compound 18 in 76% yield; mp>300° C. ¹H NMR (D₂O/DMSO-d₆): 7.88-7.99 (m, 5H), 8.10 (d, J=3.9 Hz, 1H), 8.38-8.42 (m, 2H), 9.08 (d, J=1.8 Hz, 1H). ¹³C NMR (D₂O/DMSO-d₆): 164.8, 161.3, 150.0, 146.0, 144.5, 142.0, 139.6, 135.0, 133.9, 131.0, 129.4, 128.7, 126.8, 125.7, 123.4. MS (ESI) m/e (rel. int.); 369 (M⁺, 40), 370 (M⁺+1, 100), 353 (80), 335 (15), 230 (20). Anal. Calcd. for C₁₇H₁₅N₅Se.2.0HCl.1.5H₂O: C, 43.61; H, 4.31; N, 14.94. Found: C, 43.55; H, 4.36; N, 14.55.
5-{5-[4-(N-Hydroxyamidino)-phenyl]selenophen-2-yl}-pyridine-2-N-hydroxyamidine (19). The same procedure described for 12 was followed, except starting with dinitrile 17, to furnish 19 in 97% yield; mp 220-222° C. ¹H NMR (DMSO-d₆): 5.68 (s, 2H), 5.72 (s, 2H), 7.61 (d, J=8.4 Hz, 2H), 7.68-7.71 (m, 3H), 7.78 (d, J=3.9 Hz, 1H), 7.85 (d, J=8.4 Hz, 1H), 7.99 (dd, J=8.4, 2.4 Hz, 1H), 8.79 (d, J=2.4 Hz, 1H), 9.55 (s, 1H), 9.82 (s, 1H). ¹³C NMR (DMSO-d₆): 150.1, 149.6, 149.1, 148.7, 144.5, 144.2, 135.4, 132.9, 132.6, 131.3, 128.5, 127.1, 125.8, 125.1, 119.3. MS (ESI) m/e (rel. int.); 402 (M⁺+1, 100), 240 (5), 162 (40).
5-{5-[4-(N-Methoxyamidino)-phenyl]-selenophen-2-yl}-pyridine-2-N-methoxyamidine (20). The same procedure described for 13 was used, except starting with diamidoxime 19, to afford 20 in 71% yield; mp 212-213° C. (DMF). ¹H NMR (DMSO-d₆): 3.77 (s, 3H), 3.83 (s, 3H), 5.97 (s, 2H), 6.00 (s, 2H), 7.64-7.77 (m, 5H), 7.84-7.89 (m, 2H), 8.03 (dd, J=8.4, 2.4 Hz, 1H), 8.85 (d, J=2.4 Hz, 1H). ¹³C NMR (DMSO-d₆): 150.4, 149.7, 148.9, 147.8, 144.7, 144.3, 135.8, 133.1, 131.9, 131.8, 128.8, 127.4, 126.3, 125.2, 119.8, 60.9, 60.4. MS (ESI) m/e (rel. int.); 429 (M⁺, 10), 428 (30), 411 (10), 398 (10), 379 (100). Hydrochloride salt of 20: mp 215-216.5° C. Anal. Calcd. for C₁₉H₁₉N₅O₂Se.2.0HCl.2.5H₂O: C, 41.77; H, 4.79; N, 12.80. Found. C, 42.06; H, 4.74; N, 12.51.

### Example 4

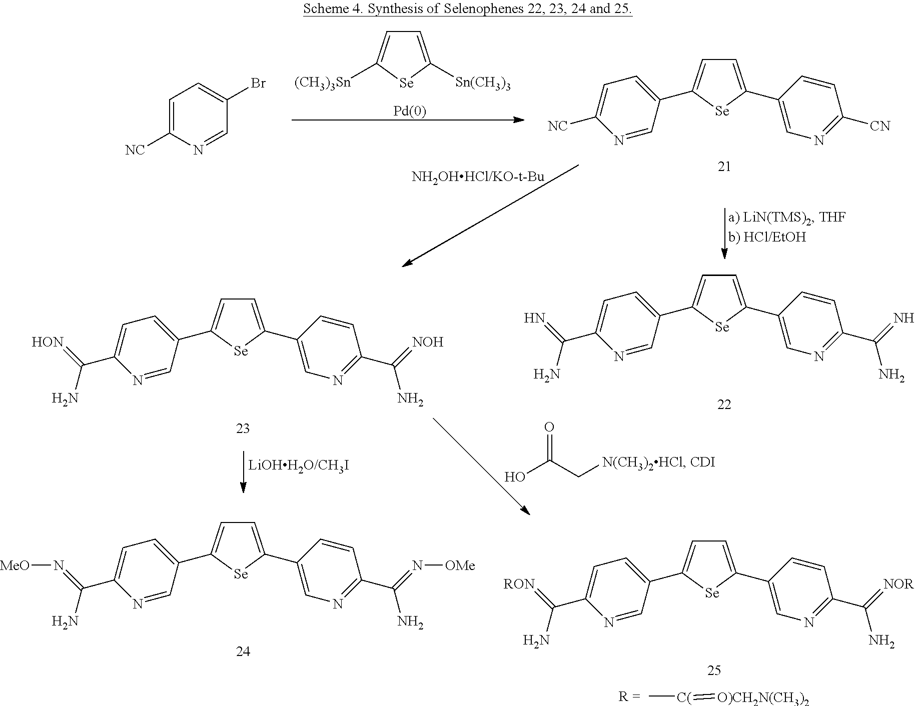

2,5-Bis-[5-(2-cyano-pyridyl)]selenophene (21). Referring now to Scheme 4 above, to a degassed mixture of 5-bromo-2-cyanopyridine (3.66 g, 0.02 mol) in 60 mL anhydrous dioxane was added Pd(PPh₃)₄ (2 mol %) followed by the addition of 2,5-bis(trimethylstannyl) selenophene. The mixture was refluxed for 10-12 h, while the reaction was followed by thin layer chromatography (TLC; 8:2 EtOAc-hexane). The solvent was removed under reduced pressure and the residue was triturated with hexanes and filtered. The resulting solid was washed with hexanes and the remaining yellow cake was dissolved in hot DMF (300 mL) to which was added 150 mL EtOAc. The hot solution was filtered through a bed of celite to remove Pd. The celite was washed with a mixture of hot DMF/EtOAc which was added to the filtered solution. The filtered solution was concentrated under vacuum, diluted with water, and filtered. The residue was then washed with water and dried under vacuum. The resulting solid was suspended in a methanol/CHCl₃ mixture (1:3, 75 mL), stirred, washed with hexanes and dried under vacuum at 80° C. for 12 h to provide a yellow solid, 2.35 g (70% yield); mp 278-80° C. dec. ¹H-NMR (DMSO-d₆): 9.05 (d, m, J=2.4 Hz), 8.25 (dd, 2H, J=8.1 Hz), 8.04 (s, 2H), 7.99 (dd, 2H, J=8.1 Hz). ¹³C-NMR (DMSO-d₆): 147.8, 146.2, 134.3, 134.0, 131.2, 131.0, 129.1, 117.3. MS: m/e 335 (M⁺). Anal. calcd. for C₁₆H₈N₄Se: C, 57.32; H, 2.40; N, 16.71. Found: C, 56.98; H, 2.64; N, 16.51.
2,5-Bis-[5-(2-amidino-pyridyl)]selenophene dihydrochloride (22). LiN(TMS)₂ (1M in THF; 10.4 mL; 0.01 mol) was added to a cooled suspension of dinitrile 21 (0.5 g; 0.0015 mol) in 10 mL anhydrous THF. The resulting mixture was stirred for 12 h or until the solution became clear. The mixture was cooled and acidified with HCl saturated EtOH and stirred for approximately 3 h. The solvent was removed, and the remaining solution was triturated with hexanes and filtered. The solid was suspended in water (40 mL), basified with 2M NaOH, filtered, washed with water, and dried over P₂O₅ under vacuum to provide 0.41 g (74%) of the free base of compound 22. The free base (0.4 g, 0.0011 mol) was suspended in anhydrous EtOH and the solution was saturated with HCl gas. The solution was stirred for 2 h, the solvent was removed, and the residue was triturated with ether. The resulting brown solid was collected by filtration, washed with ether, and dried at 60° C. under vacuum to provide 0.43 g (84%) of dihydrochloride salt 22; mp>300° C. dec. ¹H-NMR (DMSO-d₅): 9.58 (br, 4H), 9.40 (br, 4H), 9.11 (m, 2H), 8.41 (m, 4H), 8.16 (s, 2H). ¹³C-NMR (DMSO-d₆): 161.4, 146.4, 146.2, 142.5, 135.1, 134.4, 131.3, 123.7. MS: m/e 370 (M⁺+1). Anal. calcd. for C₁₆H₁₄N₆Se.2HCl.H₂O: C, 41.75; H, 3.94; N, 18.26. Found: C, 41.89; H, 3.92; N, 17.94.
2,5-Bis-[5-{2-(N-hydroxyamidino)-pyridyl}]selenophene dihydrochloride (23). To a cold suspension of hydroxylamine hydrochloride (2.78 g; 0.04 mol) in anhydrous DMSO (75 mL) was added KO-t-Bu (4.48 g; 0.04 mol); and the mixture was stirred at room temperature for 30 min, followed by the addition of dinitrile 21 (1.675 g; 0.005 mol). The resulting mixture was stirred at room temperature for 12-15 h, filtered into 200 mL of ice water and stirred. The precipitated yellow product was collected by filtration, washed with water, and dried under vacuum over P₂O₅ at 30° C. to provide 1.75 g (88%) of the free base of bis-amidoxime 23. ¹H-NMR (DMSO-d₆) (free base): δ 9.78 (s, 2H), 8.84 (d, 2H, J=2.1H), 8.03 (dd, 2H, J=2.1 Hz, J=8.4 Hz), 7.90 (d, 2H, J=8.4 Hz), 7.84 (s, 2H), 5.71 (br s, 4H). The free base (0.5 g; 0.0012 mol) was converted into its brown hydrochloride salt by stirring with ethanolic HCl in ethanol, trituration with ether and filtration to give 0.56 g (92%) of 23; mp>340° C. dec. ¹H-NMR (DMSO-d₆/D₂O) (salt): 8.99 (d, 2H, J=2.1 Hz), 8.26 (dd, 2H, J=2.1 Hz, J=8.4 Hz), 8.17 (d, 2H, J=8.4 Hz), 8.03 (s, 2H), 6.4-5.4 (vbr, residual exchangeable protons). ¹³C-NMR (DMSO-d₆): 153.6, 145.4, 143.8, 139.8, 133.4, 131.5, 128.0, 122.3. MS: m/e 402 (M⁺+1). Anal. calcd. for 23: C₁₆H₁₄N₆O₂Se.2HCl.0.75H₂O: C, 39.4; H, 3.61; N, 17.23. Found: C, 39.73; H, 3.65; N, 16.89.
2,5-Bis-[5-{2-(N-methoxyamidino)-pyridyl}]selenophene dihydrochloride (24). To a cold degassed suspension of bis-amidoxime 23 (0.5 g; 0.00125 mol) in 35 mL anhydrous DMF was added 0.157 g (0.0037 mol) LiOH.H₂O in 2 mL water. After stirring for 2 h the amidoxime mixture was added over 10 min to a solution of CH₃I (0.64 g; 0.0044 mol) in 2 mL DMF. The amidoxime/CH₃I reaction mixture was allowed to stir for 24 h or until clear. The reaction mixture was then poured into ice water. The precipitated yellow solid was filtered, washed with water, and dried under vacuum over P₂O₅ at 30° C. to give the free base of 24 (0.44 g; 86%). ¹H-NMR (DMSO-d₆) (free base): 8.85 (d, 2H, J=2.1H), 8.05 (dd, 2H, J=2.1 Hz, J=8.4 Hz), 7.88 (d, 2H, J=8.4 Hz), 7.87 (s, 2H), 5.96 (br s, 4H), 3.83 (s, 6H). ¹³C-NMR (DMSO-d₆) (free base): 148.8, 148.0, 145.6, 144.8, 133.2, 131.5, 128.8, 119.7, 0.8. The free base (0.4 g, 0.0009 mol) was converted to its hydrochloride salt (0.42 g; 92%); mp>280° C. dec. ¹H-NMR (DMSO-d₆/D₂O) (salt): 8.87 (d, 2H, J=1.5 Hz), 8.09 (dd, 2H, J=1.5 Hz, J=8.7 Hz), 7.94 (d, 2H, J=8.7 Hz), 7.89 (s, 2H), 5.24 (br, 6H), 3.85 (s, 6H). ¹³C-NMR (DMSO-d₆) (salt): 150.2, 145.8, 145.2, 133.6, 132.3, 129.5, 123.5, 120.8, 61.6. MS: m/e 430 (M⁺+1). Anal. calcd. for C₁₈H₁₈N₆O₂Se.2HCl.H₂O: C, 41.55; H, 4.26; N, 16.15. Found: C, 41.22; H, 4.32; N, 16.17.
2,5-Bis-[5-{2-N—(N,N-dimethylaminoacetoxyamidino)-pyridyl}]selenophene (25). N,N′-Carbonyldiimidazole (CDI; 0.81 g; 0.005 mol) was added to a stirred mixture of N,N-dimethylaminoacetic acid hydrochloride (0.7 g; 0.005 mol) in 10 mL DMF and heated at 60° C. for 2 h. The mixture was then cooled to 0° C., followed by the addition of bis-amidoxime 23 (0.5 g; 0.00125 mol). This mixture was stirred for 12 h at room temperature, during which the clear solution turned cloudy. After 12 h the mixture was poured into an ice/salt mixture (approximately 100 mL), which was stirred in ice for 2-3 h, and filtered. The precipitated yellow solid was collected by filtration, washed with water, and dried under vacuum over P₂O₅ to give compound 25 in 77% yield (0.56 g). ¹H-NMR (DMSO-d₆): 8.95 (d, 2H, J=1.8 Hz), 8.18 (dd, 2H, J=1.8 Hz, J=8.4 Hz), 7.99 (s, 2H), 7.98 (d, 2H, J=8.4 Hz), 6.84 (br, 4H), 3.38 (s, 4H), 2.32 (s, 12H). ¹³C-NMR (DMSO-d₆): 167.3, 153.7, 146.8, 145.0, 133.5, 132.7, 121.0, 58.0, 44.2. MS: m/e 571 (M⁺). Anal. calcd. for C₂₄H₂₈N₈O₄Se.0.5H₂O: C, 49.56; H, 5.04; N, 19.30. Found: C, 49.27; H, 5.22; N, 19.11.

### Example 5

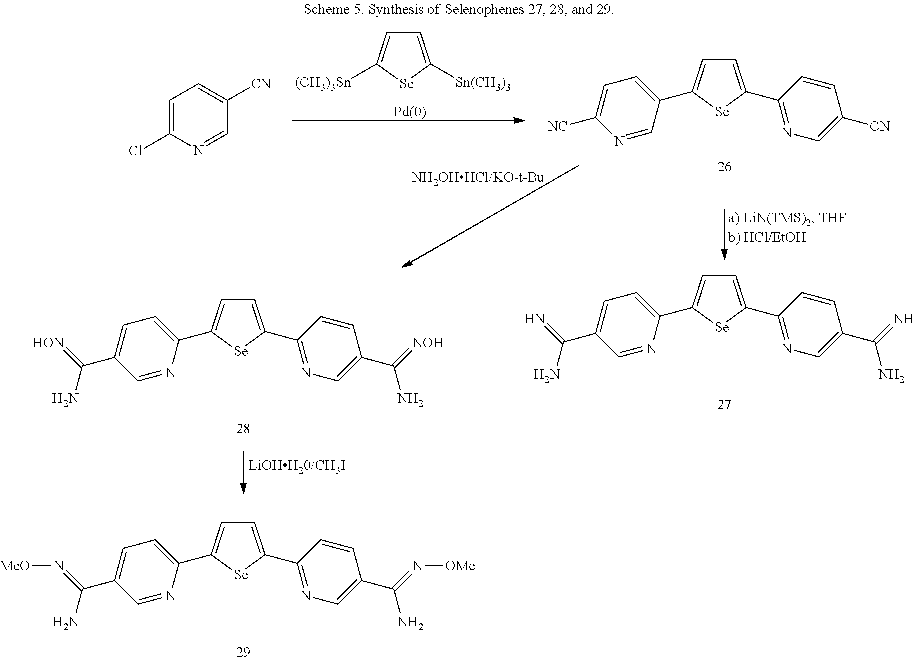

2,5-Bis-[2-(5-cyano-pyridyl)]selenophene (26). Referring now to Scheme 5 above, the reaction of 2-chloro-5-cyanopyridine 2.77 g, (0.02 mol), 2,5-bis(trimethylstannyl)selenophene and Pd(PPh₃)₄ (2 mol %) in 60 mL anhydrous dioxane was performed in a manner analogous to that described for the preparation of compound 21. Work-up yielded 2.6 g (77%) of yellow solid 26; mp>320° C. dec. ¹H-NMR (DMSO-d₆): 8.92 (brs, 2H), 8.28 (d, 2H, J=8.4 Hz), 8.23 (s, 2H), 8.13 (d, 2H, J=8.4 Hz). ¹³C-NMR (DMSO-d₆): 155.5, 152.5, 152.4, 140.4, 131.2, 117.8, 116.7, 107.0. MS: m/e 335 (M⁺). Anal. calcd. for C₁₆H₈N₄Se: C, 57.32; H, 2.40; N, 16.71. Found: C, 57.03; H, 2.40; N, 16.39.
2,5-Bis-[2-(5-amidino-pyridyl)]selenophene dihydrochloride (27). The reaction of dinitrile 26 (0.335 g; 0.001 mol) and LiN(TMS)₂ (1M in THF; 7 mL; 0.0071 mol) in 10 mL anhydrous THF was performed analogous to that described for compound 22, above. Workup yielded the free base of the title compound as a yellow solid (0.3 g; 81% yield). The free base (0.29 g; 0.00079 mol) was suspended in anhydrous EtOH and saturated with HCl gas and stirred for 2 h. The solvent was removed and the residue was triturated with ether. The resulting brown solid was collected by filtration, washed with ether and dried at 60° C. under vacuum to give 0.31 g (88% yield) of dark yellow hydrochloride salt 27; mp>330° C. dec. ¹H-NMR (DMSO-d₆/D₂O) (salt): 891 (dd, 2H, J=0.9 Hz, J=2.4 Hz), 8.27 (s, 2H), 8.24 (dd, 2H, J=2.4 Hz, J=8.4 Hz), 8.18 (dd, 2H, J=0.9 Hz, J=8.4 Hz). ¹³C-NMR (DMSO-d₆/D₂O): 163.7; 157.0, 152.7, 149.2, 137.3, 131.4, 122.8, 117.8. MS: m/e 370 (M⁺+1). Anal. calcd. for C₁₆H₁₄N₆Se.2HCl.0.25H₂O: C, 43.02; H, 3.72; N, 18.81. Found: C, 43.32; H, 3.78; N, 18.55.
2,5-Bis-[2-{5-(N-hydroxyamidino)-pyridyl}]selenophene dihydrochloride (28). The reaction of the dinitrile 26 (1.675 g; 0.005 mol) and hydroxylamine (prepared by reacting NH₂OH.HCl (2.78 g; 0.04 mol) and KO-t-Bu (4.48 g; 0.04 mol) in 75 mL anhydrous DMSO) was performed according to a procedure analogous to that described above for the preparation of compound 23. After work-up, the product was dried under vacuum over P₂O₅ at 30° C. to provide 1.79 g (89% yield) of the free base of compound 28. The free base (0.5 g; 0.0012 mol) was converted into its brown hydrochloride salt by stirring with ethanolic HCl, trituration with ether, and filtration to yield 0.55 g (93%) of 28; mp>260° C. dec. ¹H-NMR (DMSO-d₆): 9.79 (s, 2H), 8.77 (d, 2H, J=1.87 Hz), 8.02 (dd, 2H, J=1.8 Hz, J=8.4 Hz), 8.0 (s, 2H), 7.92 (d, 2H, J=8.4 Hz), 5.89 (vbr, 6H). ¹³C-NMR (DMSO-d₆): 156.5, 156.1, 152.4, 148.6, 136.8, 130.9, 120.6, 117.5; MS: m/e 402 (M⁺+1). Anal. calcd. for: C₁₆H₁₄N₆O₂Se.2HCl.H₂O: C, 39.04; H, 3.69; N, 17.07. Found: C, 39.02; H, 3.55; N, 16.76.
2,5-Bis-[2-{5-(N-methoxyamidino)-pyridyl}]selenophene dihydrochloride (29). To a cold degassed suspension of bis-amidoxime 28 (0.5 g; 0.00125 mol) in 35 mL anhydrous DMF was added LiOH.H₂O (0.157 g; 0.0037 mol) in 2 mL water. After this mixture was stirred for 2 h, a solution of CH₃I (0.64 g; 0.0044 mol) in 2 mL DMF was added over 10 min and the resulting mixture was allowed to stir for 24 h or until clear. The reaction mixture was poured into an ice/water mixture, and the precipitated yellow solid was filtered, washed with water, and dried under vacuum over P₂O₅ at 30° C. to provide 0.41 g (76% yield) of the free base of 29. The free base (0.4 g; 0.00093 mol) was converted to its hydrochloride salt (0.43 g; 87%); mp>280° C. dec. ¹H-NMR (DMSO-d₆): 8.84 (d, 2H, J=1.5 Hz), 8.13 (dd, 2H, J=1.5 Hz, J=8.7 Hz), 8.09 (s, 2H), 8.0 (d, 2H, J=8.7 Hz), 5.86 (vbr, 6H), 3.84 (s, 6H). ¹³C-NMR (DMSO-d₆): 154.4, 152.1, 151.6, 147.3, 135.0, 129.5, 117.2, 61.6. MS: m/e 430 (M⁺+1). Anal. calcd. for C₁₈H₁₈N₆O₂Se.2HCl.1.75H₂O: C, 40.50; H, 4.43; N, 15.74. Found: C, 40.44; H, 4.33; N, 15.95.

### Example 6

### Di-azaaryl Thiophene Compounds

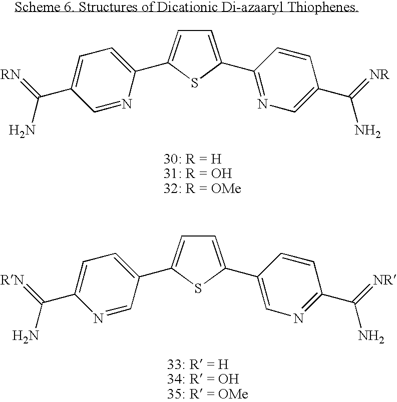

Dicationic di-azaaryl thiophene compounds having the structures shown above in Scheme 6 were prepared according to procedures analogous to those used to prepare the di-azaaryl selenophenes (e.g., 22, 23, 24, 25, 27, 28, and 29) as described hereinabove in Examples 4 and 5, except that a bis-alkylstannyl thiophene was used in place of the 2,5-bis(trimethylstannyl)selenophene.

2,5-Bis-[2-(5-cyanopyridyl)]thiophene. Reaction of 2-chloro-5-cyanopyridine (2.77 g, 0.02 mol), 2,5-bis(trimethylstannyl)thiophene (4.09 g, 0.01 mol) and Pd(PPh₃)₄ (2 mol %) in 60 mL anhydrous dioxane under N₂ reflux yielded, after work-up, the title dinitrile as a yellow solid, 2.2 g (76% yield); m.p.>320° C. dec. ¹H-NMR (DMSO-d₆, 80° C.): 8.96 (d, 2H, J=2.1 Hz), 8.30 (dd, 2H, J=2.1 Hz, J=8.4 Hz), 8.13 (d, 2H, J=8.4 Hz), 8.03 (s, 2H). MS: m/e 288 (M⁺). Anal. calcd. for C₁₆H₈N₄S: C, 66.65; H, 2.79; N, 19.43. Found: C, 66.49; H, 2.82; N, 19.36.
2,5-Bis-[2-(5-amidino-pyridyl)]thiophene dihydrochloride (30). Reaction of 2,5-bis-[2-(5-cyanopyridyl)]thiophene (0.5 g, 0.00174 mol) and LiN(TMS)₂ (1M solution in THF, 12 mL, 0.012 mol) in 10 mL anhydrous THF followed by workup yielded the yellow solid free base of 30 in 75% yield (0.42 g). The free base (0.4 g, 0.0012 mol) was suspended in anhydrous EtOH, triturated with HCl gas, and stirred for 2 h. Solvent was removed, and the residue was triturated with ether. The resultant brown solid was filtered, washed with ether, and dried at 60° C. under vacuum to give 0.4 g (82% yield) dark yellow hydrochloride salt; m.p.>310° C. dec. ¹H-NMR (DMSO-d₆) (salt): 9.46 (br, 4H), 9.19 (br, 4H), 9.04 (d, 2H, J=2.4 Hz), 9.31 (dd, 2H, J=2.4 Hz, J=8.7 Hz), 8.20 (d, 2H, J=8.7 Hz), 8.07 (s, 2H). ¹³C-NMR (DMSO-d₆): 163.4, 155.0, 148.8, 146.0, 136.9, 128.8, 122.5, 118.2. MS: m/e 323 (M⁺+1).
2,5-Bis-[2-{5-(N-hydroxyamidino)-pyridyl}]thiophene dihydrochloride (31). The title compound was prepared by reaction of 2,5-bis-[2-(5-cyanopyridyl)]thiophene (1.3 g; 0.0045 mol) and hydroxylamine (prepared by reacting NH₂OH.HCl (2.5 g; 0.036 mol) and potassium tert-butoxide (4.0 g; 0.036 mol) in 75 mL anhydrous DMSO. Work-up and drying in vacuum over P₂O₅ at 30° C. gave 1.35 g (85%) free base. The free base (0.45 g; 0.0013 mol) was converted into its brown hydrochloride salt by stirring with ethanolic HCl in ethanol and triturated with ether and filtered to yield 0.48 g 31 (83% yield); m.p.>275° C. dec. ¹H-NMR (DMSO-d₆): 9.17 (s, 2H), 8.42 (s, 2H, J=8.4 Hz), 8.21 (d, 2H, J=8.4 Hz), 7.94 (s, 2H). ¹³C-NMR (DMSO): 154.7, 145.4, 144.7, 139.8, 135.4, 132.5, 127.9, 119.2. MS: m/e 402 (M⁺+1). Anal. calcd. for: C₁₆H₁₄N₆O₂S.2HCl.1.25H₂O: C, 45.62; H, 3.93; N, 17.73. Found: C, 45.69; H, 4.11; N, 17.61.
2,5-Bis-[2-{5-(N-methoxyamidino)-pyridyl}]thiophene dihydrochloride (32). To a cold degassed suspension of 31 (0.5 g, 0.0014 mol) in 30 mL anhydrous DMF was added LiOH.H₂O (0.176 g, 0.0042 mol) in 2 mL water. After stirring for 2 h, a solution of CH₃I (0.64 g, 0.0044 mol) in 2 mL DMF was added over 10 min. The entire mixture was allowed to stir for 24 hr or until clear. The reaction mixture was poured in ice-water and the precipitated yellow solid was filtered, washed with water, and dried in vacuum over P₂O₅ at 30° C. to provide the free base of 32 (0.34 g, 69% yield). The free base (0.3 g, 0.00078 mol) was converted to its hydrochloride salt (0.31 g, 82% yield); m. p.>280° C. dec. ¹H-NMR (DMSO-d₆): 8.97 (s, 2H), 8.21 (dd, 2H, J=2.1 Hz, J=8.7 Hz), 7.99 (d, 2H, J=8.7 Hz), 7.98 (s, 2H), 5.62 (vbr, 6H, exchangeable), 3.85 (s, 6H). ¹³C-NMR (DMSO-d₆) (salt): 153.0, 144.7, 141.7, 134.4, 127.7, 124.3, 122.2, 121.6, 61.4. MS: m/e 430 (M⁺+1). Anal. calcd. for C₁₈H₁₈N₆O₂S.2HCl.1.5H₂O: C, 44.81; H, 4.80; N, 17.42. Found: C, 44.76; H, 4.93; N, 17.49.
2,5-Bis-[5-(2-cyano-pyridyl)]thiophene. To a degassed mixture of 5-bromo-2-cyanopyridine (3.66 g, 0.02 mol) and 2,5-bis(trimethylstannyl)thiophene (4.90 g, 0.01 mol) in 60 mL anhydrous dioxane was added Pd(PPh₃)₄ (2 mol %) and the mixture was refluxed for 10-12 hr (TLC followed, 8:2 EtOAc:hexanes). Solvent was removed and the residue triturated with hexane, filtered, and then washed with hexane. The yellow cake with Pd was dissolved in approximately 400 mL hot DMF. Ethyl acetate (50 mL) was added and the hot solution was filtered through a celite bed which was then washed with a mixture of hot DMF:EtOAc (3:1, 100 mL). The solvent was concentrated in vacuum, diluted with water and filtered, washed with water, and dried in vacuum. The solid was suspended in a methanol:CHCl₃ (1:3, 75 mL) mixture, stirred, washed with hexane and dried in vacuum at 80° C. for 12 hr to provide the title dinitrile as a yellow solid (2.1 g, 73% yield); m.p.>290° C. dec. ¹H-NMR (DMSO-d₆): 9.20 (d, 2H, J=2.1 Hz), 8.32 (dd, 2H, J=2.1 Hz, J=8.4 Hz), 8.11 (d, 2H, J=8.4 Hz), 7.83 (s, 2H). ¹³C-NMR (DMSO-d₆): 146.8, 145.8, 134.2, 133.2, 130.6, 129.8, 128.5, 117.3. MS: m/e 288 (M⁺). Anal. calcd. for C₁₆H₈N₄S: C, 66.65; H, 2.79; N, 19.43. Found: C, 66.61; H, 2.73; N, 19.33.
2,5-Bis-[5-(2-amidino-pyridyl)]thiophene dihydrochloride (33). LiN(TMS)₂ (1M solution in THF, 12 mL, 0.012 mol) was added to a cooled suspension of 2,5-bis-[5-(2-cyano-pyridyl)]thiophene (0.5 g, 0.00174 mol) in 10 mL anhydrous THF. The mixture was stirred for 12 hr or until the solution became clear, then cooled and acidified with HCl-saturated EtOH and stirred for approximately 3 h. The solvent was removed, and the residue triturated with hexane and filtered. The resulting solid was suspended in water (40 mL), basified with 2M NaOH, filtered, washed with water, and dried over P₂O₅ in vacuum to give 0.42 g (75% yield) of the free base of 33. The free base (0.4 g, 0.0012 mol) was suspended in anhydrous EtOH, triturated with HCl gas, and stirred for 2 h. The solvent was removed, and the residue was triturated with ether and the resulting brown solid filtered, washed with ether and dried at 60° C. in vacuum to provide 0.41 g (85% yield) of hydrochloride salt 33; m.p.>300° C. dec. ¹H-NMR (DMSO-d₆): 9.24 (br, 4H), 6.0 (s, 2H), 8.09 (vbr, 4H), 8.06 (d, 2H, J=8.4 Hz), 8.12 (d, 2H, J=8.4 Hz), 7.76 (s, 2H). ¹³C-NMR (DMSO-d₆): 162.2, 147.2, 142.7. 141.0, 134.9, 134.1, 129.7, 124.2. MS: m/e 323 (M⁺+1). Anal. calcd. for C₁₆H₁₄N₆S.2HCl.0.5H₂O: C, 47.53; H, 4.24; N, 20.76. Found: C, 47.58; H, 4.33; N, 20.88.
2,5-Bis-[5-{2-(N-hydroxyamidino)-pyridyl}]thiophene dihydrochloride (34). To a cold suspension of hydroxylamine-hydrochloride (2.5 g; 0.036 mol) in anhydrous DMSO (75 mL) was added KO-t-Bu (4.0 g, 0.036 mol) and the mixture was stirred at room temperature for 30 min, followed by the addition of 2,5-bis-[5-(2-cyano-pyridyl)]thiophene (1.30 g, 0.0045 mol). The entire mixture was stirred at room temperature for 12-15 hr, filtered into 200 mL ice-water mixture, stirred, and the precipitated yellow product was filtered, washed with water and dried in vacuum over P₂O₅ at 30° C. to give 1.3 g (82% yield) of the free base of 34. The free base (0.45 g, 0.0013 mol) was converted into its brown hydrochloride salt by stirring with ethanolic-HCl in ethanol, triturated with ether, and filtered to yield 0.47 g (84% yield) of 34; m.p.>340° C. dec. ¹H-NMR (DMSO-d₆/D₂O) (salt): 9.07 (s, 2H), 8.32 (s, 2H, J=8.4 Hz), 8.18 (d, 2H, J=8.4 Hz), 7.92 (s, 2H). ¹³C-NMR (DMSO-d₆/D₂O): 153.7, 145.4, 143.7, 139.8, 133.4, 131.5, 127.9, 122.2. MS: m/e 355 (M⁺+1). Anal. calcd. for: C₁₆H₁₄N₆O₂S.2HCl.0.75H₂O: C, 43.59; H, 4.00; N, 19.06. Found: C, 43.62; H, 4.19; N, 18.89.
2,5-Bis-[5-{2-(N-methoxyamidino)-pyridyl}]thiophene dihydrochloride (35). To a cold degassed suspension of 34 (0.5 g, 0.0014 mol) in 30 mL anhydrous DMF was added LiOH.H₂O (0.176 g, 0.0042 mol) in 2 mL water. After stirring for 2 h, a solution of CH₃I (0.6 g, 0.0042 mol) in 2 mL DMF was added over 10 minutes, and the entire mixture was allowed to stir for 24 hr or until clear. The reaction mixture was poured in ice-water, and the precipitated yellow solid was filtered, washed with water, and dried in vacuum over P₂O₅ at 30° C. to provide 0.35 g (71% yield) of the free base of 35. The free base (0.31 g, 0.00078 mol) was converted to its hydrochloride salt (0.33 g, 82% yield); m. p.>280° C. dec. ¹H-NMR (DMSO-d₆) (salt): 8.95 (s, 2H), 8.15 (dd, 2H, J=2.1 Hz, J=8.7 Hz), 7.99 (d, 2H, J=8.7 Hz), 7.78 (s, 2H), 5.56 (vbr, 6H, exchangeable), 3.85 (s, 6H). ¹³C-NMR (DMSO-d₆) (salt): 150.0, 144.7, 139.7, 133.1, 127.1, 123.3, 121.0, 120.6, 61.4. MS: m/e 383 (M⁺+1). Anal. calcd. for C₁₈H₁₈N₆O₂S.2HCl.3H₂O: C, 42.43; H, 5.14; N, 16.49. Found: C, 42.55; H, 5.32; N, 16.27.

### Example 7

### In Vitro Antiprotozoan Activity of Dicationic Diaryl Thiophenes and Selenophenes

In vitro antiprotozoal activities were measured following established protocols. See Ismail, M. A., et al., J. Med. Chem., 46, 4761-4769 (2003); Stephens, C. E., et al., Bioorg. Med. Chem. Lett., 13, 2065-2069 (2003) (in vitro assay against Leishmania donovani). The activities of compounds 4, 11, 18, 22, 27, 30 and 33 against Trypanosoma brucei rhodesiense (T. b. r.), Plasmodium falciparum (P. f.), Leishmania donovani (L. d.), and L-6 rat mycoblast cells (as an assay for cell toxicity) are shown in Table 1. These values are compared to those of furamidine, pentamidine, a di-amidino-diphenyl thiophene (compound 36) and a diamidino diaryl thiophene compound containing a single pyridyl group (compound 37).

The presently disclosed compounds show good activity against T.b.r., P.f., and L.d. in vitro. Compounds 4, 11, 18, 22, and 33 show IC₅₀ values versus T.b.r. of 26 nm or less. Compounds 4, 11, 18, 22, 27, and 33 all show IC₅₀ values versus P.f. of 14 nm or less (i.e., less than that of furamidine). The data versus L. d. was particularly promising as several compounds (i.e., 4, 18, 22, and 33) show IC₅₀ values versus L.d. of less than 0.2 μM, which is significantly less than that of the standard drug pentamidine. Compound II also shows an IC₅₀ value versus L.d. of 1.6 μM, which is slightly less than that shown by pentamidine and furamidine.

### Example 8

### In Vivo Antiprotozoan Activity of Dicationic Diaryl Thiophenes and Selenophenes

The activities of compounds 4, 5, 6, 11, 12, 18, 20, 22, 27, 28, 30 and 33 against the STIB 900 strain of Trypanosoma brucei rhodesiense (T. b. r.) in a mouse model are shown in Table 2. These values are compared to those of pentamidine, furamidine, DB 351 and DB 1214. Groups of four mice were infected intraperitoneally with 2×10⁵ bloodstream forms of T. b. r. STIB 900 which originates from a patient in Tanzania. On days 3, 4, 5, and 6 post-infection the experimental groups were treated with the drugs either by the intraperitoneal or for prodrugs by the oral route. Usually the highest tolerated dose was used which was determined in a pretoxicological experiment. Parasitemia of the mice was checked daily up to day 14 post-infection and thereafter 2 times per week up to day 60. One group of mice was not treated and acted as control. For relapsing mice, the day of death was recorded and the survival time determined.

The presently disclosed compounds have good activity against T.b.r. in vivo. Four diamidine compounds (i.e., 11, 18, 22, and 27) show 4/4 cures versus the STIB 900 strain of T.b.r. in the mouse model, compared to the 0/4 cures shown by furamidine and pentamidine. Compound 4 shows 2/4 cures, while compounds 30 and 33 both show 1/4 cures. The prodrugs of the presently disclosed compounds also show promise as orally effective agents against T.b.r. In particular, prodrug 20 shows 1/4 cures against the mouse T.b.r. model.

TABLE 1
In Vitro Activity of Dicationic Selenophene and Thiophene
Derivatives.

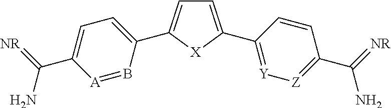

| | | | | | | | T.b.r. | P.f. | L.d. | Cyto- |
|---|---|---|---|---|---|---|---|---|---|---|
| Com- | | | | | | | IC₅₀ | IC₅₀ | C₅₀ | toxocity |
| pound | X | A | B | Y | Z | R | (nM) | (nM) | (μM) | (μM) |
| Penta- | NA | NA | NA | NA | NA | NA | 2.2 | NT | 2.0 | 11.4 |
| midine | | | | | | | | | | |
| Furami- | O | CH | CH | CH | CH | H | 4.3 | 15.5 | 2.7 | 6.4 |
| dine | | | | | | | | | | |
| 36 | S | CH | CH | CH | CH | H | 2.4 | 13.7 | 0.42 | 51.7 |
| 37 | S | CH | N | CH | CH | H | 13 | 11 | 3.4 | 42.6 |
| 30 | S | CH | N | N | CH | H | 68 | 18 | 23.3 | >222 |
| 33 | S | N | CH | CH | N | H | 4 | 3 | 0.127 | 4.8 |
| 4 | Se | CH | CH | CH | CH | H | 2 | 2 | 0.17 | 9.7 |
| 18 | Se | N | CH | CH | CH | H | 26 | 14 | 0.13 | 13.4 |
| 11 | Se | CH | N | CH | CH | H | 9 | 10 | 1.6 | 27.7 |
| 22 | Se | N | CH | CH | N | H | 2 | 4 | 0.053 | 5.6 |
| 27 | Se | CH | N | N | CH | H | 50 | 10 | 5.6 | 158.3 |

NA = not applicable
Pentamidine:

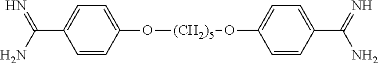

TABLE 2
In Vivo Activity of Dicationic Selenophene and Thiophene Derivatives.

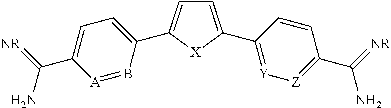

| | | | | | | | | Dosage | | Survival |
|---|---|---|---|---|---|---|---|---|---|---|
| Compound | X | A | B | Y | Z | R | Dosage Route^{a} | (mg/kg) | Cures^{b} | (days)^{c} |
| Pentamidine | NA | NA | NA | NA | NA | NA | ip | 20 | 0/4 | 42.75 |
| Furamidine | O | CH | CH | CH | CH | H | ip | 10 | 0/4 | 44.5 |
| 36 | S | CH | CH | CH | CH | H | ip | 20 | 2/4 | 42.75 |
| 37 | S | CH | N | CH | CH | H | ip | 20 | 2/4 | 42.5 |
| 30 | S | CH | N | N | CH | H | ip | 5 | 1/4 | 50.25 |
| 33 | S | N | CH | CH | N | H | ip | 5 | 1/4 | 42.5 |
| 4 | Se | CH | CH | CH | CH | H | ip | 20 | 2/4 | 55.5 |
| 5 | Se | CH | CH | CH | CH | OH | po | 25 | 0/4 | 11.5 |
| 6 | Se | CH | CH | CH | CH | OMe | po | 25 | 0/4 | 15.25 |
| 11 | Se | CH | N | CH | CH | H | ip | 5 | 4/4 | 60 |
| 12 | Se | CH | N | CH | CH | OH | po | 25 | 0/4 | 26 |
| 18 | Se | N | CH | CH | CH | H | ip | 5 | 4/4 | 60 |
| 20 | Se | N | CH | CH | CH | OMe | po | 25 | 1/4 | 36.5 |
| 22 | Se | N | CH | CH | N | H | ip | 5 | 4/4 | 60 |
| 27 | Se | CH | N | N | CH | H | ip | 5 | 4/4 | 60 |
| 28 | Se | CH | N | N | CH | OH | po | 25 | 0/4 | 20 |

NA = not applicable
^{a}ip = intraperitoneal; po = oral
^{b}Number of mice that survive and are parasite free for 60 days
^{c}Average days of survival; untreated control animals expire between 7 and 8 days post infection

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A compound having a structure of Formula (I): 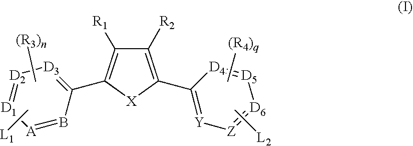
wherein:
X is Se or S;
D₁, D₂, D₃, D₄, D₅, and D₆ are each independently C or N;
A, B, Y, and Z are each independently selected from the group consisting of CH and N, provided that when X is S, at least two of A, B, Y, and Z are N;
n is an integer from 0 to 2;
q is an integer from 0 to 2;
R₁ and R₂ are independently selected from the group consisting of H, alkyl, substituted alkyl, cycloalkyl, aryl, substituted aryl, aralkyl, halogen, alkoxyl, aryloxyl, aralkoxyl, and hydroxyl;
each R₃ and R₄ is independently selected from the group consisting of H, alkyl, substituted alkyl, cycloalkyl, aryl, substituted aryl, aralkyl, halogen, alkoxyl, aryloxyl, aralkoxyl, and hydroxyl; and
L₁ and L₂ are independently selected from the group consisting of: 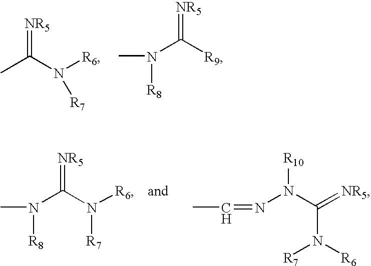
wherein:
each R₅ is selected from the group consisting of H, hydroxyl, alkyl, cycloalkyl, aryl, aralkyl, alkoxyl, aryloxyl, hydroxycycloalkyl, alkoxycycloalkyl, hydroxyalkyl, aminoalkyl, acyloxyl, and alkylaminoalkyl; and
each R₆, R₇, R₈, R₉, and R₁₀ is independently selected from the group consisting of H, alkyl, alkoxyalkyl, cycloalkyl, aryl, aralkyl, hydroxyalkyl, aminoalkyl, and alkylaminoalkyl, or a R₅ and a R₆ or a R₅ and a R₉ together represent a C₂ to C₁₀ alkylene, or a R₅ and a R₆ or a R₅ and a R₉ together are: 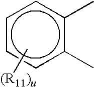
wherein u is an integer from 1 to 4, and R₁₁ is H or —CONHR₁₂NR₁₃R₁₄, wherein R₁₂ is alkylene and R₁₃ and R₁₄ are each independently selected from the group consisting of H, alkyl, aryl and aralkyl; or
a pharmaceutically acceptable salt thereof.

2. The compound of claim 1, wherein the compound of Formula (I) has a structure of Formula (Ia): 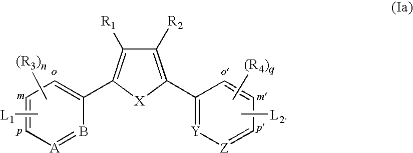
wherein:
X is Se or S;
A, B, Y, and Z are each independently selected from the group consisting of CH and N, provided that when X is S, at least two of A, B, Y, and Z are N;
n is an integer from 0 to 2;
q is an integer from 0 to 2;
R₁ and R₂ are independently selected from the group consisting of H, alkyl, substituted alkyl, cycloalkyl, aryl, substituted aryl, aralkyl, halogen, alkoxyl, aryloxyl, aralkoxyl, and hydroxyl;
each R₃ and R₄ is independently selected from the group consisting of H, alkyl, substituted alkyl, cycloalkyl, aryl, substituted aryl, aralkyl, halogen, alkoxyl, aryloxyl, aralkoxyl, and hydroxyl; and
L₁ and L₂ are independently selected from the group consisting of: 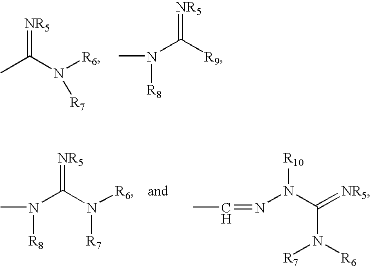
wherein:
each R₅ is selected from the group consisting of H, hydroxyl, alkyl, cycloalkyl, aryl, aralkyl, alkoxyl, aryloxyl, hydroxycycloalkyl, alkoxycycloalkyl, hydroxyalkyl, aminoalkyl, acyloxyl, and alkylaminoalkyl; and
each R₆, R₇, R₈, R₉, and R₁₀ is independently selected from the group consisting of H, alkyl, alkoxyalkyl, cycloalkyl, aryl, aralkyl, hydroxyalkyl, aminoalkyl, and alkylaminoalkyl, or a R₅ and a R₆ or a R₅ and a R₉ together represent a C₂ to C₁₀ alkylene, or a R₅ and a R₆ or a R₅ and a R₉ together are: 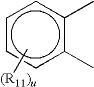
wherein u is an integer from 1 to 4, and R₁₁ is H or —CONHR₁₂NR₁₃R₁₄, wherein R₁₂ is alkylene and R₁₃ and R₁₄ are each independently selected from the group consisting of H, alkyl, aryl and aralkyl; or
a pharmaceutically acceptable salt thereof.

3. The compound of claim 2, wherein L₁ is attached at the p carbon and L₂ is attached at the p′ carbon.

4. The compound of claim 1, wherein L₁ and L₂ are each: 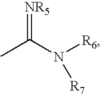
wherein:
each R₅ is selected from the group consisting of H, hydroxyl, alkoxyl, and acyloxyl; and
each R₆ and R₇ are independently selected from the group consisting of H, lower alkyl, cycloalkyl, aryl, aralkyl, hydroxyalkyl, aminoalkyl, and alkylaminoalkyl, or a R₅ and a R₆ together represent a C₂ to C₁₀ alkylene.

5. The compound of claim 4, wherein each R₅ is selected from the group consisting of H, hydroxyl, methoxyl and N,N-dimethylaminoacetoxyl.

6. The compound of claim 4, wherein each R₆ and R₇ is H.

7. The compound of claim 1, wherein compound of Formula (I) has a structure of Formula (II): 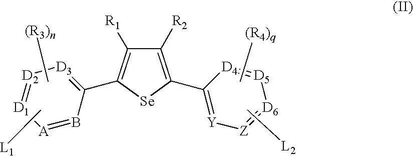
wherein:
D₁, D₂, D₃, D₄, D₅, and D₆ are each independently C or N;
A, B, Y, and Z are each independently selected from the group consisting of CH and N;
n is an integer from 0 to 2;
q is an integer from 0 to 2;
R₁ and R₂ are independently selected from the group consisting of H, alkyl, substituted alkyl, cycloalkyl, aryl, substituted aryl, aralkyl, halogen, alkoxyl, aryloxyl, aralkoxyl, and hydroxyl;
each R₃ and R₄ is independently selected from the group consisting of H, alkyl, substituted alkyl, cycloalkyl, aryl, substituted aryl, aralkyl, halogen, alkoxyl, aryloxyl, aralkoxyl, and hydroxyl; and
L₁ and L₂ are independently selected from the group consisting of: 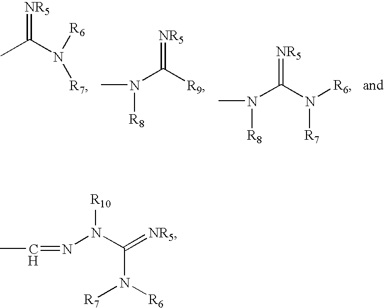
wherein:
each R₅ is selected from the group consisting of H, hydroxyl, alkyl, cycloalkyl, aryl, aralkyl, alkoxyl, aryloxyl, hydroxycycloalkyl, alkoxycycloalkyl, hydroxyalkyl, aminoalkyl, acyloxyl, and alkylaminoalkyl; and
each R₆, R₇, R₈, R₉, and R₁₀ is independently selected from the group consisting of H, alkyl, alkoxyalkyl, cycloalkyl, aryl, aralkyl, hydroxyalkyl, aminoalkyl, and alkylaminoalkyl, or a R₅ and a R₆ or a R₅ and a R₉ together represent a C₂ to C₁₀ alkylene, or a R₅ and a R₆ or a R₅ and a R₉ together are: 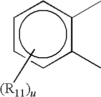
wherein u is an integer from 1 to 4, and R₁₁ is H or —CONHR₁₂NR₁₃R₁₄, wherein R₁₂ is alkylene and R₁₃ and R₁₄ are each independently selected from the group consisting of H, alkyl, aryl and aralkyl; or
a pharmaceutically acceptable salt thereof.

8. The compound of claim 7, wherein the compound of Formula (II) has a structure of Formula (IIa): 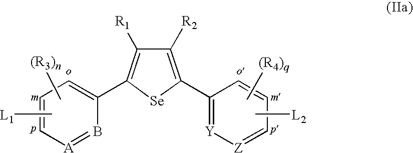
wherein:
A, B, Y, and Z are each independently selected from the group consisting of CH and N;
n is an integer from 0 to 2;
q is an integer from 0 to 2;
R₁ and R₂ are independently selected from the group consisting of H, alkyl, substituted alkyl, cycloalkyl, aryl, substituted aryl, aralkyl, halogen, alkoxyl, aryloxyl, aralkoxyl, and hydroxyl;
each R₃ and R₄ is independently selected from the group consisting of H, alkyl, substituted alkyl, cycloalkyl, aryl, substituted aryl, aralkyl, halogen, alkoxyl, aryloxyl, aralkoxyl, and hydroxyl; and
L₁ and L₂ are independently selected from the group consisting of: 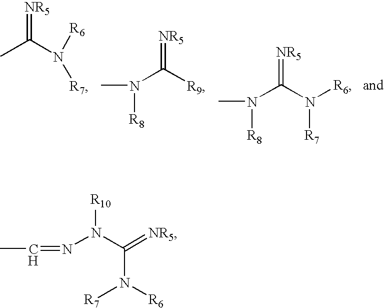
wherein:
each R₅ is selected from the group consisting of H, hydroxyl, alkyl, cycloalkyl, aryl, aralkyl, alkoxyl, aryloxyl, hydroxycycloalkyl, alkoxycycloalkyl, hydroxyalkyl, aminoalkyl, acyloxyl, and alkylaminoalkyl; and
each R₆, R₇, R₈, R₉, and R₁₀ is independently selected from the group consisting of H, alkyl, alkoxyalkyl, cycloalkyl, aryl, aralkyl, hydroxyalkyl, aminoalkyl, and alkylaminoalkyl, or a R₅ and a R₆ or a R₅ and a R₉ together represent a C₂ to C₁₀ alkylene, or a R₅ and a R₆ or a R₅ and a R₉ together are: 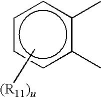
wherein u is an integer from 1 to 4, and R₁₁ is H or —CONHR₁₂NR₁₃R₁₄, wherein R₁₂ is alkylene and R₁₃ and R₁₄ are each independently selected from the group consisting of H, alkyl, aryl and aralkyl; or
a pharmaceutically acceptable salt thereof.

9. The compound of claim 7, wherein A, B, Y, and Z are each CH.

10. The compound of claim 7, wherein at least one of A, B, Y, and Z is N.

11. The compound of claim 10, wherein B and Y are each N.

12. The compound of claim 10, wherein A and Z are each N.

13. The compound of claim 1, wherein the compound of Formula (I) has a structure of Formula (III): 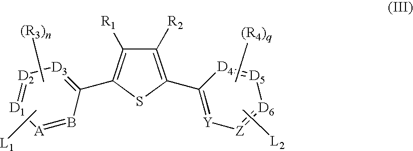
wherein:
D₁, D₂, D₃, D₄, D₅, and D₆ are each independently C or N;
A, B, Y, and Z are each independently selected from the group consisting of CH and N, provided that at least two of A, B, Y, and Z are N;
n is an integer from 0 to 2;
q is an integer from 0 to 2;
R₁ and R₂ are independently selected from the group consisting of H, alkyl, substituted alkyl, cycloalkyl, aryl, substituted aryl, aralkyl, halogen, alkoxyl, aryloxyl, aralkoxyl, and hydroxyl;
each R₃ and R₄ is independently selected from the group consisting of H, alkyl, substituted alkyl, cycloalkyl, aryl, substituted aryl, aralkyl, halogen, alkoxyl, aryloxyl, aralkoxyl, and hydroxyl; and
L₁ and L₂ are independently selected from the group consisting of: 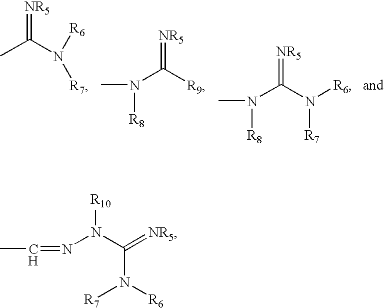
wherein:
each R₅ is selected from the group consisting of H, hydroxyl, alkyl, cycloalkyl, aryl, aralkyl, alkoxyl, aryloxyl, hydroxycycloalkyl, alkoxycycloalkyl, hydroxyalkyl, aminoalkyl, acyloxyl, and alkylaminoalkyl; and
each R₆, R₇, R₈, R₉, and R₁₀ is independently selected from the group consisting of H, alkyl, alkoxyalkyl, cycloalkyl, aryl, aralkyl, hydroxyalkyl, aminoalkyl, and alkylaminoalkyl, or a R₅ and a R₆ or a R₅ and a R₉ together represent a C₂ to C₁₀ alkylene, or a R₅ and a R₆ or a R₅ and a R₉ together are: 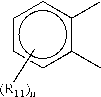
wherein u is an integer from 1 to 4, and R₁₁ is H or —CONHR₁₂NR₁₃R₁₄, wherein R₁₂ is alkylene and R₁₃ and R₁₄ are each independently selected from the group consisting of H, alkyl, aryl and aralkyl; or
a pharmaceutically acceptable salt thereof.

14. The compound of claim 13, wherein D₁, D₂, D₃, D₄, D₅, and D₆ are each C.

15. The compound of claim 14, wherein the compound of Formula (III) has a structure of one of Formula (IIIb) and Formula (IIIc): 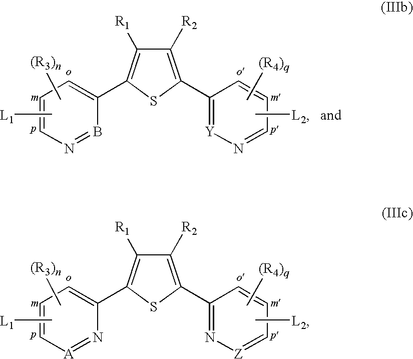
wherein:
A, B, Y, and Z are each independently selected from the group consisting of CH and N;
n is an integer from 0 to 2;
q is an integer from 0 to 2;
R₁ and R₂ are independently selected from the group consisting of H, alkyl, substituted alkyl, cycloalkyl, aryl, substituted aryl, aralkyl, halogen, alkoxyl, aryloxyl, aralkoxyl, and hydroxyl;
each R₃ and R₄ is independently selected from the group consisting of H, alkyl, substituted alkyl, cycloalkyl, aryl, substituted aryl, aralkyl, halogen, alkoxyl, aryloxyl, aralkoxyl, and hydroxyl; and
L₁ and L₂ are independently selected from the group consisting of: 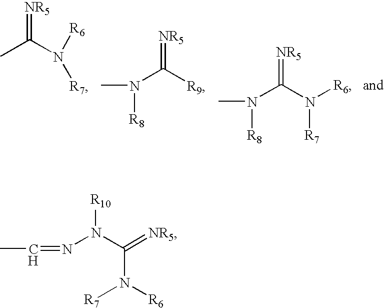
wherein:
each R₅ is selected from the group consisting of H, hydroxyl, alkyl, cycloalkyl, aryl, aralkyl, alkoxyl, aryloxyl, hydroxycycloalkyl, alkoxycycloalkyl, hydroxyalkyl, aminoalkyl, acyloxyl, and alkylaminoalkyl; and
each R₆, R₇, R₈, R₉, and R₁₀ is independently selected from the group consisting of H, alkyl, alkoxyalkyl, cycloalkyl, aryl, aralkyl, hydroxyalkyl, aminoalkyl, and alkylaminoalkyl, or a R₅ and a R₆ or a R₅ and a R₉ together represent a C₂ to C₁₀ alkylene, or a R₅ and a R₆ or a R₅ and a R₉ together are: 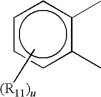
wherein u is an integer from 1 to 4, and R₁₁ is H or —CONHR₁₂NR₁₃R₁₄, wherein R₁₂ is alkylene and R₁₃ and R₁₄ are each independently selected from the group consisting of H, alkyl, aryl and aralkyl; or
a pharmaceutically acceptable salt thereof.

16. The compound of claim 1, wherein the compound of Formula (I) is selected from the group consisting of:
2,5-Bis(4-amidinophenyl)selenophene;
2,5-Bis[4-(N-hydroxyamidino)phenyl]selenophene;
2,5-Bis[4-(N-methoxyamidino)phenyl]selenophene;
6-[5-(4-amidinophenyl)-selenophen-2-yl]-nicotinamidine;
6-{5-[4-(N-hydroxyamidino)phenyl]-selenophen-2-yl}-N-hydroxynicotinamidine;
6-{5-[4-(N-methoxyamidino)phenyl]-selenophen-2-yl}-N-methoxynicotinamidine;
5-[5-(4-amidinophenyl)-selenophene-2-yl]-pyridine-2-amidine;
5-{5-[4-(N-hydroxyamidino)-phenyl]-selenophen-2-yl}-pyridine-2-N-hydroxamidine;
5-{5-[4-(N-methoxyamidino)-phenyl]-selenophen-2-yl}-pyridine-2-N-methoxamidine;
2,5-Bis-[5-(2-amidinopyridyl)]selenophene;
2,5-Bis-[5-{2-(N-hydroxyamidino)-pyridyl}]selenophene;
2,5-Bis-[5-{2-(N-methoxyamidino)-pyridyl}]selenophene;
2,5-Bis-[5-{2-N—(N,N-dimethylaminoacetoxyamidino)-pyridyl}]selenophene;
2,5-Bis-[2-(5-amidinopyridyl)]selenophene;
2,5-Bis-[2-{5-(N-hydroxyamidino)-pyridyl}]selenophene;
2,5-Bis-[2-{5-(N-methoxyamidino)pyridyl}]selenophene;
2,5-Bis-[2-(5-amidinopyridyl)]thiophene;
2,5-Bis-[2-{5-(N-hydroxyamidino)-pyridyl}]thiophene;
2,5-Bis-[2-{5-(N-methoxyamidino)-pyridyl}]thiophene;
2,5-Bis-[5-(2-amidinopyridyl)]thiophene;
2,5-Bis-[5-{2-(N-hydroxyamidino)-pyridyl}]thiophene; and
2,5-Bis-[5-{2-(N-methoxyamidino)-pyridyl}]thiophene;
or a pharmaceutically acceptable salt thereof.

17. The compound of claim 1, wherein the pharmaceutically acceptable salt is a hydrochloride salt.

18. A pharmaceutical formulation comprising:
a compound of claim 1; and
a pharmaceutically acceptable carrier.

19. A method of treating a microbial infection in a subject in need of treatment thereof, the method comprising administering to the subject an effective amount of a compound of Formula (I): 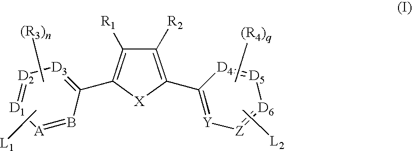
wherein:
X is Se or S;
D₁, D₂, D₃, D₄, D₅, and D₆ are each independently C or N;
A, B, Y, and Z are each independently selected from the group consisting of CH and N, provided that when X is S, at least two of A, B, Y, and Z are N;
n is an integer from 0 to 2;
q is an integer from 0 to 2;
R₁ and R₂ are independently selected from the group consisting of H, alkyl, substituted alkyl, cycloalkyl, aryl, substituted aryl, aralkyl, halogen, alkoxyl, aryloxyl, aralkoxyl, and hydroxyl;
each R₃ and R₄ is independently selected from the group consisting of H, alkyl, substituted alkyl, cycloalkyl, aryl, substituted aryl, aralkyl, halogen, alkoxyl, aryloxyl, aralkoxyl, and hydroxyl; and
L₁ and L₂ are independently selected from the group consisting of: 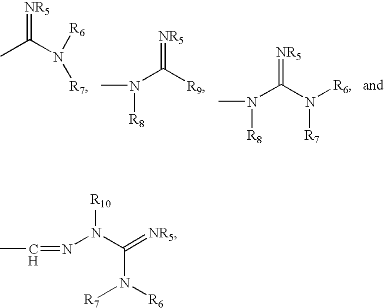
wherein:
each R₅ is selected from the group consisting of H, hydroxyl, alkyl, cycloalkyl, aryl, aralkyl, alkoxyl, aryloxyl, hydroxycycloalkyl, alkoxycycloalkyl, hydroxyalkyl, aminoalkyl, acyloxyl, and alkylaminoalkyl; and
each R₆, R₇, R₈, R₉, and R₁₀ is independently selected from the group consisting of H, alkyl, alkoxyalkyl, cycloalkyl, aryl, aralkyl, hydroxyalkyl, aminoalkyl, and alkylaminoalkyl, or a R₅ and a R₆ or a R₅ and a R₉ together represent a C₂ to C₁₀ alkylene, or a R₅ and a R₆ or a R₅ and a R₉ together are: 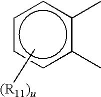
wherein u is an integer from 1 to 4, and R₁₁ is H or —CONHR₁₂NR₁₃R₁₄, wherein R₁₂ is alkylene and R₁₃ and R₁₄ are each independently selected from the group consisting of H, alkyl, aryl and aralkyl; or
a pharmaceutically acceptable salt thereof.

20. The method of claim 19, wherein the microbial infection is selected from the group consisting of a Trypanosoma brucei rhodesiense infection, a Plasmodium falciparum infection, and a Leishmania donovoni infection.

21. The method of claim 19, wherein the compound of Formula (I) is administered prophylactically to prevent or reduce the incidence of one of:
a microbial infection in a subject at risk of infection;
a recurrence of the microbial infection; and
combinations thereof.

22. The method of claim 19, wherein X is Se.

23. The method of claim 19, wherein the compound of Formula (I) is selected from the group consisting of:
2,5-Bis(4-amidinophenyl)selenophene;
2,5-Bis[4-(N-hydroxyamidino)phenyl]selenophene;
2,5-Bis[4-(N-methoxyamidino)phenyl]selenophene;
6-[5-(4-amidinophenyl)-selenophen-2-yl]-nicotinamidine;
6-{5-[4-(N-hydroxyamidino)phenyl]-selenophen-2-yl}-N-hydroxynicotinamidine;
6-{5-[4-(N-methoxyamidino)phenyl]-selenophen-2-yl}-N-methoxynicotinamidine;
5-[5-(4-amidinophenyl)-selenophene-2-yl]-pyridine-2-amidine;
5-{5-[4-(N-hydroxyamidino)-phenyl]-selenophen-2-yl}-pyridine-2-N-hydroxamidine;
5-{5-[4-(N-methoxyamidino)-phenyl]-selenophen-2-yl}-pyridine-2-N-methoxamidine;
2,5-Bis-[5-(2-amidinopyridyl)]selenophene;
2,5-Bis-[5-{2-(N-hydroxyamidino)-pyridyl}]selenophene;
2,5-Bis-[5-{2-(N-methoxyamidino)-pyridyl}]selenophene;
2,5-Bis-[5-{2-N—(N,N-dimethylaminoacetoxyamidino)-pyridyl}]selenophene;
2,5-Bis-[2-(5-amidinopyridyl)]selenophene;
2,5-Bis-[2-{5-(N-hydroxyamidino)-pyridyl}]selenophene;
2,5-Bis-[2-{5-(N-methoxyamidino)pyridyl}]selenophene;
2,5-Bis-[2-(5-amidinopyridyl)]thiophene;
2,5-Bis-[2-{5-(N-hydroxyamidino)-pyridyl}]thiophene;
2,5-Bis-[2-{5-(N-methoxyamidino)-pyridyl}]thiophene;
2,5-Bis-[5-(2-amidinopyridyl)]thiophene;
2,5-Bis-[5-{2-(N-hydroxyamidino)-pyridyl}]thiophene; and
2,5-Bis-[5-{2-(N-methoxyamidino)-pyridyl}]thiophene;
or a pharmaceutically acceptable salt thereof.
